(19) United States
(12) Patent Application Publication
(10) Pub. No.: US 2026/0170640 A1
(43) Pub. Date: Jun. 18, 2026

(54) RETINAL SCAN IMAGE CLASSIFICATION

(71) Applicant: UCL BUSINESS LTD, London (GB)

(72) Inventors: Nikolas PONTIKOS, London (GB); William WOOF, London (GB)

(73) Assignee: UCL BUSINESS LTD, London (GB)

(21) Appl. No.: 19/122,352

(22) PCT Filed: Sep. 26, 2023
(86) PCT No.: PCT/EP2023/076614

§ 371 Date: Apr. 18, 2025

(30) Foreign Application Priority Data
Oct. 21, 2022 (GB) 2215626.9

(51) Int. Cl. G06T 7/00; G06V 40/18; G16H 30/40; G16H 50/20; G16H 50/30

(52) U.S. Cl. CPC G06T 7/0012; G06V 40/197; G16H 30/40; G16H 50/20; G16H 50/30; G06T 2207/10101; G06T 2207/20084; G06T 2207/30041

(57) ABSTRACT

A computer-implemented method of retinal scan image classification, the method comprising: receiving an input dataset comprising at least one retinal scan, the retinal scan acquired using one of a plurality of imaging modalities; passing the input dataset through an ensemble network, the ensemble network comprising a plurality of [trained] convolutional neural networks, wherein each convolutional neural network is configured to classify retinal scans acquired using one of the plurality of imaging modalities based on a plurality of eye pathoses, each convolutional neural network producing probabilities of the presence of each of the eye pathoses within retinal scans, and the retinal scan is processed using a convolutional neural network corresponding to the imaging modality used for acquisition of the retinal scan; and producing ensembled probabilities of the presence of each of the eye pathoses within the retinal scan.

### FIELD OF THE INVENTION

The present invention relates to methods of retinal scan image classification, neural networks for use in retinal scan image classification, and methods of training networks for use in retinal scan image classification. In addition, the present invention relates to data processing apparatuses, computer programs, and computer-readable media for retinal scan image classification. In particular, the present invention relates to methods and networks for estimating probabilities of the presence of a plurality of eye pathoses within retinal scans.

### BACKGROUND OF THE INVENTION

Inherited retinal diseases (IRDs) are a group of rare genetic conditions affecting 1 in 3000 people, with over 300 different IRD-associated genes identified to date. IRDs cause deterioration of the retina, the light-sensitive tissue at the back of the eye, responsible for vision. Some patients with IRDs may be profoundly visually impaired from birth, while others may find that their peripheral and/or central vision progressively deteriorates over time. Cumulatively, IRDs are a leading cause of blindness in children and the most common cause in the working-age population, with a significant psychological and socioeconomic impact.

Revealing the genetic cause of an IRD is a prerequisite to optimally determining prognosis, providing genetic counselling, eligibility for gene-directed treatments, and inclusion in gene-directed clinical trials. However, this genetic diagnosis remains elusive in more than 40% of cases, largely due to lack of access to services or inefficiencies in diagnostic services such as insufficient evidence, clinical experience, and the ability to successfully link or communicate data and findings between research groups. In many parts of the world, almost 100% of cases are not genetically solved, due to lack of access and resources to fund molecular testing.

IRDs can have distinct phenotypic features that clinicians learn to recognise using modern retinal imaging technology that rapidly and non-invasively acquires images of the retina via a dilated pupil with minimal inconvenience to the patient. These scans can be performed via a variety of imaging modalities such as fundus autofluorescence (FAF), infrared (IR) imaging, and spectral-domain optical coherence tomography (SD-OCT), each of which convey different information about the retinal architecture.

FIG. 1 provides example retinal scans (also referred to as eye or ophthalmic scans or images) acquired using these three example imaging modalities. Panel A is an IR fundus scan acquired at 30 degrees. Panel B is a FAF scan acquired at 55 degrees. Panel C is a single B-scan from an SD-OCT volume.

For example, FAF images can help identify patterns of both photoreceptor dysfunction and apoptosis by specifically detecting lipofuscin and related compounds by their autofluorescent properties, which primarily accumulate in the retinal pigment epithelium (RPE) promoted by oxidative stress. IR imaging helps identify vascular abnormalities but also melanin, a naturally occurring pigment that protects the eye, and melanin lipofuscin, a granule containing both melanin and lipofuscin. SD-OCT helps identify and characterize the multiple cellular layers of the retina such as the RPE, photoreceptors, outer nuclear and plexiform, inner nuclear and plexiform, ganglion cells, and nerve fibre layer. The principal cells affected by IRDs are the photoreceptors, with SD-OCT able to delineate the hyperreflective ellipsoid zone (EZ) which is used to identify photoreceptor inner and outer segment layers and is often disrupted early in IRDs.

This high-resolution, in-depth multimodal information enables ophthalmologists to identify some gene-specific patterns of disease, allowing prediction of the disease-associated gene in some cases. However, given the sparsity of these diseases, the expertise and experience required to make accurate clinical diagnoses is not widely available and is limited to a handful of specialists and hospitals who have developed this expertise over several decades.

This limitation is particularly evident when considering the diagnosis of IRDs; a growing number are now being targeted in clinical trials, with approved treatment now available. However, access requires a genetic diagnosis to be established sufficiently early. Critically, the timely identification of a genetic cause remains challenging.

The inventors have come to the realisation that timely identification of eye pathoses, in particular eye pathoses with a genetic determinant, is therefore desirable.

### SUMMARY OF THE INVENTION

According to an aspect of the invention, there is provided a computer-implemented method of retinal scan image classification. The method comprises a step of receiving an input dataset comprising at least one retinal scan, the retinal scan acquired using one of a plurality of imaging modalities. That is, following acquisition of a retinal scan (or perhaps multiple retinal scans), the classification method involves acceptance of the retinal scan(s), for processing. This may be, for example, directly via the acquisition machine or may be through the user uploading the retinal scan(s) for processing.

The method further comprises passing the input dataset through an ensemble network, the ensemble network comprising a plurality of convolutional neural networks (CNNs). Each CNN here is configured to classify retinal scans acquired using one of the plurality of imaging modalities based on a plurality of eye pathoses. That is, a first CNN is configured to classify—based on eye pathoses, for example pathoses with a genetic basis—retinal scans acquired using a first imaging modality, and a second CNN is configured to classify—based on eye pathoses—retinal scans acquired using a second imaging modality, etc. Each CNN produces probabilities of the presence of each of the eye pathoses within retinal scans. The retinal scan(s) (passed into the ensemble network) is (are) processed using a CNN corresponding to the imaging modality used for acquisition of the retinal scan(s). For instance, if the retinal scan(s) is (are) are acquired using a first imaging modality, the retinal scan(s) are processed using the CNN corresponding to the first imaging modality. The CNNs are trained. A CNN (or a group thereof) for a single imaging modality may be referred to as a predictor block or a modality-specific ensemble model.

The CNNs may be configured to output probabilities for a predefined number of classes, the number of classes corresponding to the number of pathoses under investigation. For example, if the ensemble network is to classify retinal scans in respect of 10 classes, 10 probabilities will be output from the ensemble network.

Ensembled probabilities are not intended to replace genetic testing, as phenotyping can never completely replace molecular testing, especially when a treatment such as gene therapy is to be administered based on a genetic diagnosis. Rather, retinal scan image classification techniques facilitate access to diagnostic expertise, only currently available in a limited number of specialist centres globally, and thereby dramatically accelerate the genetic diagnostic odyssey. Use of CNNs for classification of eye pathoses is a promising approach to accelerating medical diagnoses, for example genetic diagnosis for patients with IRD, especially given the growing number of treatable IRDs where a rapid diagnosis can lead to an improved outcome for the patient.

The method further comprises producing ensembled (or aggregated) probabilities of the presence of each of the eye pathoses within the retinal scan. In the event that only a single retinal scan is passed through the ensemble network, and there exists only a single CNN for the imaging modality used for acquisition of the single retinal scan, the ensembled probabilities are the probabilities produced from the CNN. In the event that only a single retinal scan is passed through the ensemble network, and there exist multiple CNNs within a modality-specific predictor block for the imaging modality used for acquisition of the single retinal scan, the ensembled probabilities may be the average of the probabilities produced from the multiple CNNs. The ensembled probabilities may be output in the form of a list of probabilities, one for each class of classification.

Optionally, the input dataset may comprise a plurality of retinal scans, each retinal scan acquired using one of the plurality of imaging modalities. In this case, each retinal scan may be processed using a CNN (or multiple CNNs within a modality-specific predictor block) corresponding to the imaging modality used for acquisition of the retinal scan. For instance, the input dataset may include at least one retinal scan from each of the imaging modalities. The input dataset may include at least one retinal scan from each of two imaging modalities. That is, in the event that a first retinal scan, acquired using a first imaging modality, and a second retinal scan, acquired using a second imaging modality, are passed through the ensemble network, the ensembled probabilities are some function (e.g., average) of the probabilities produced from a first CNN (for the first imaging modality) and a second CNN (the second imaging modality). Using multiple retinal scans of the same imaging modality increases the confidence of eye pathoses classification of the CNN(s) for that imaging modality. Using multiple retinal scans of different imaging modalities similarly increases the confidence of eye pathoses classification.

Optionally, the plurality of imaging modalities may comprise any or all of the following: Fundus autofluorescence, FAF; infrared, IR; and spectral-domain optical coherence tomography, SD-OCT. These are imaging modalities commonly used for the acquisition of retinal scans. Ensemble networks built on CNNs corresponding to these imaging modalities are found to be successful in identifying eye pathoses. For instance, due to transformational improvements in imaging technology and a comprehensive genetic testing framework for IRDs embedded in specialist healthcare services over the last decade, there are now a sufficient number of molecularly characterized patients with detailed retinal phenotyping (using, e.g., the above imaging modalities to build representative datasets for deep learning). Incidentally, FAF and IR scans may be acquired at varying angular spreads, for example 30 degrees and 55 degrees; these may be considered as the same or distinct imaging modalities.

Optionally, the plurality of eye pathoses are genetic abnormalities (or mutations). In this way, the ensemble network is well-suited for aiding in detection of rare eye diseases such as IRDs, which are otherwise challenging to diagnose genetically. IRDs are typically monogenic disorders and represent a leading cause of blindness in children and working-age adults worldwide. Wider access to expertise previously restricted to human specialists in the field may then be offered via an AI system trained to detect gene-specific patterns from retinal scans. Even when restricted to a single imaging modality, the accuracy of the ensemble network, when classifying genetic abnormalities, performs at least as well, and often significantly better, than human experts at this task.

Optionally, the method may further comprise passing the ensembled probabilities through a trained linear classifier. This supplements or refines the ensembled probabilities output from the CNN(s) on the basis of further information encoded into the classifier. For instance, the linear classifier may be configured to refine the ensembled probabilities based on subject age and/or mode of inheritance, MOI, of genetic abnormalities. Use of a linear classifier is shown to improve classification accuracy. Other factors, such as ethnicity of subject, may additionally or alternatively be encoded within the trained linear classifier.

Optionally, the ensemble network may comprise a number (k) of CNNs per imaging modality. The retinal scan is processed using k CNNs, each CNN corresponding to the imaging modality used for acquisition of the retinal scan. That is, multiple trained CNNs for a single imaging modality may simultaneously be applied to a single retinal scan. The method may then involve a step of averaging the probabilities of the presence of each of the eye pathoses within the retinal scans from each of the k CNNs. The averaged probabilities (of which there may be multiple, one set for each group of CNNs) may then be used to produce the ensembled probabilities. Using multiple CNNs per imaging modality is shown to improve accuracy of classification relative to use of a single CNN.

Optionally, the method may further comprise outputting, when the estimation of the presence of each of the eye pathoses within the retinal scan is below a predefined confidence threshold, a prompt to reacquire the retinal scan. For instance, where the training dataset used to train the CNNs is sparsely populated (for instance, with retinal scans for rare pathoses), trained CNNs may be prone to overpredict the most common classes and underpredict the rarer ones in the presence of uncertainty; introducing a threshold then introduces further certainty to retinal scan image classifications. Further, lower quality retinal scans can also confound the results, especially if linked to age. For example, retinal scans in younger children tend to be noisier, in part due to poorer compliance or movement during image acquisition; use of a confidence threshold quickly flags such scans to the user.

Optionally, the method may further comprise processing the output dataset to provide a classification of genetic cause of eye pathosis. For example, where the eye pathosis is a genetic-based pathosis, the method accelerates the time to diagnosis because it can point to a specific gene to be investigated. The method will aid the vast majority of ophthalmologists and other vision healthcare workers who are not specialized in rare IRDs to indicate when molecular testing would be worthy of consideration. Genetic predictions may also be used for scoring and therefore prioritizing genetic variants where the gene matches the phenotype. In disorders with a highly distinct phenotype, the “matching phenotype” PP4 criterion in the ACMG guidelines provides a higher level of evidence in variant classification, which may be crucial for the transition from a variant of unknown significance to a likely pathogenic variant.

According to an aspect of the invention, there is provided an ensemble network for retinal scan image classification, in particular for use in the above method of retinal scan image classification, the ensemble network comprising a plurality of CNNs. Each CNN is configured to classify retinal scans acquired using one of a plurality of imaging modalities based on a plurality of eye pathoses, each CNN producing probabilities of the presence of each of the eye pathoses within retinal scans.

According to an aspect of the invention, there is provided a computer-implemented method of training an ensemble network for retinal scan image classification, in particular the above ensemble network. The training method comprises receiving an input training dataset comprising training retinal scans labelled with acquisition imaging modality and known eye pathoses, each training retinal scan acquired using one of a plurality of imaging modalities. In one example, the training dataset may be a combination of retinal scans and a lookup table (e.g., a CSV document) referring to each of the retinal scans along with various file metadata (for example, patient.id, laterality, date, file.name, scan.id, scan.number, gene, modality, etc.).

The training method comprises splitting the training retinal scans into a plurality of sets of training retinal scans, each set of training retinal scans comprising training retinal scans acquired using the same imaging modality. Splitting may further produce a held-out validation set; this set is not to be used in actual training, but instead for quantifying the efficacy of the trained CNNs.

The training method comprises, for each set of training retinal scans, training at least one CNN so as to minimise errors between predicted classifications of a plurality of eye pathoses and the known eye pathoses. Training may be from scratch (that is, e.g., starting with a CNN architecture with randomised weights and biases), or may involve fine-tuning existing weights for a CNN (e.g., starting with ImageNet weights). Fine-tuning avoids excessive computational costs.

The training method comprises, for each CNN, outputting the trained CNN weights. That is, after training (e.g., when a predefined number of epochs has passed, or when training losses meet a predefined criterion), the CNN is “frozen”, and the checkpoint or weights are saved. The output weights may be stored locally or transmitted elsewhere, such that the retinal scan image classification method may be implemented locally or elsewhere without the need to perform training each time.

Optionally, the training method may comprise wherein training the CNNs uses k-fold cross validation, such that the method comprises, for each set of training retinal scans, training k CNNs. It is shown that training multiple networks for each imaging modality results in improved classification accuracy relative to just a single network for each. The value k in this instance may be determined in accordance with the training dataset at hand; suitable values in include 2, 5, 10, 20, or n, where n is the size of the dataset for each testo sample (leave-one-out cross-validation). Of course, the number of CNNs for one imaging modality need not be the same as the number for a second imaging modality.

Optionally, the training method may further comprise augmenting the input training dataset. For instance, the training dataset may be augmented by any or all of the following: rotating; flipping; zooming; cropping; adjusting brightness; blurring; and adding noise. Of course, the skilled reader will appreciate that other augmentation techniques are available and suitable. By augmenting the training dataset, one may increase the size and diversity of the dataset and, in turn, counter certain biases in the training data. In this way, the training method may avoid overfitting.

Optionally, the training method may further comprise pre-processing the input training dataset. For instance, the training dataset may be pre-processed by any or all of the following: filtering by median pixel intensity; filtering by noise level; filtering by identification of image artefacts; and filtering by BRISQUE score. Of course, the skilled reader will appreciate that other pre-processing techniques are available and suitable.

Embodiments of another aspect include a data processing apparatus, which comprises means suitable for carrying out a method of retinal scan image classification according to an embodiment or a method of training an ensemble network for retinal scan image classification according to an embodiment. Pre-processing removes low quality and defective training images, thereby resulting in more accurate trained CNNs.

Embodiments of another aspect include a computer program comprising instructions, which, when the program is executed by a computer, cause the computer to carry out a method of retinal scan image classification according to an embodiment or a method of training an ensemble network for retinal scan image classification according to an embodiment. The computer program may be stored on a computer-readable medium. The computer-readable medium may be non-transitory.

Hence embodiments of another aspect include a non-transitory computer-readable medium comprising instructions, which, when the program is executed by a computer, cause the computer to carry out a method of retinal scan image classification according to an embodiment or a method of training an ensemble network for retinal scan image classification according to an embodiment.

The invention may be implemented in digital electronic circuitry, or in computer hardware, firmware, software, or in combinations thereof. The invention may be implemented as a computer program or computer program product, i.e., a computer program tangibly embodied in a non-transitory information carrier, e.g., in a machine-readable storage device, or in a propagated signal, for execution by, or to control the operation of, one or more hardware modules.

A computer program may be in the form of a stand-alone program, a computer program portion or more than one computer program and may be written in any form of programming language, including compiled or interpreted languages, and it may be deployed in any form, including as a stand-alone program or as a module, component, subroutine, or other unit suitable for use in a data processing environment. A computer program may be deployed to be executed on one module or on multiple modules at one site or distributed across multiple sites and interconnected by a communication network.

Method steps of the invention may be performed by one or more programmable processors executing a computer program to perform functions of the invention by operating on input data and generating output. Apparatus of the invention may be implemented as programmed hardware or as special purpose logic circuitry, including e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions coupled to one or more memory devices for storing instructions and data.

The invention is described in terms of particular embodiments. Other embodiments are within the scope of the following claims. For example, the steps of the invention may be performed in a different order and still achieve desirable results. Multiple test script versions may be edited and invoked as a unit without using object-oriented programming technology; for example, the elements of a script object may be organized in a structured database or a file system, and the operations described as being performed by the script object may be performed by a test control program.

Elements of the invention have been described using the terms “processor”, “input device” etc. The skilled person will appreciate that such functional terms and their equivalents may refer to parts of the system that are spatially separate but combine to serve the function defined. Equally, the same physical parts of the system may provide two or more of the functions defined. For example, separately defined means may be implemented using the same memory and/or processor as appropriate.

### BRIEF DESCRIPTION OF THE DRAWINGS

Reference is made, by way of example only, to the accompanying drawings in which:

FIG. 1 is a composite image of retinal scans acquired using three imaging modalities commonly used to examine the retinas of patients with inherited retinal disease;

FIG. 2 is a flow diagram providing a method of retinal scan image classification according to an embodiment;

FIG. 3 is a diagram illustrating the contribution of each of fifteen CNNs in an example image classification method, using three imaging modalities;

FIG. 4 is a diagram illustrating an example of 5-fold cross-validation for training an ensemble network;

FIG. 5 is a plot of training loss over 100 training epochs in an example training process;

FIG. 6 is an overview of a pre-processing filtering process, used to filter an example training dataset;

FIG. 7 is an overview of data augmentations, used to augment an example training dataset;

FIG. 8 is a set of Receiver Operating Characteristic (ROC) curves acquired from cross-validation data for the three different imaging modalities across 6 of 36 predicted genes in an example;

FIG. 9 is a diagram illustrating the four distinct approaches to retinal scan image classification considered during performance evaluation;

FIG. 10 is a set of ROC curves acquired from cross-validation data for the combined prediction using an ensemble network, overlaid with the predictions for three different imaging modalities across 6 of 36 predicted genes in an example;

FIG. 11 is a calibration chart for single retinal scan processed using an ensemble network;

FIG. 12 is a diagram illustrating the distribution of subject ages at earliest presentation of genetic diagnosis in an example training dataset;

FIG. 13 is a diagram illustrating the known mode of inheritance per gene, in an example training dataset;

FIG. 14 is a set of ROC curves acquired from cross-validation data for linear classifiers trained on different combinations of features, across 6 of 36 predicted genes in an example;

FIG. 15 is a representative screen of a graphical user interface illustrating 6 retinal scans as input into an ensemble network and resultant ensembled probabilities of gene abnormalities; and

FIG. 16 is a diagram of suitable hardware for implementation of invention embodiments.

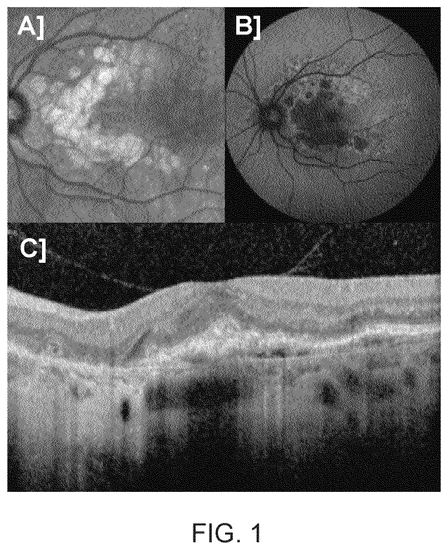

### DETAILED DESCRIPTION

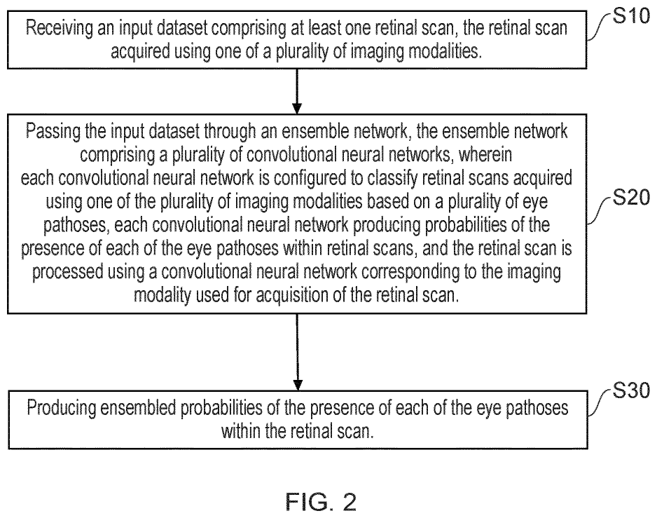

FIG. 2 is a flow chart depicting a computer-implemented method of retinal scan image classification.

At S10, the computer receives an input dataset comprising at least one retinal scan, the retinal scan acquired using one of a plurality of imaging modalities.

At S20, the computer passes the input dataset through an ensemble network, the ensemble network comprising a plurality of CNNs. Each CNN is configured to classify retinal scans acquired using one of the plurality of imaging modalities based on a plurality of eye pathoses, each CNN producing probabilities of the presence of each of the eye pathoses within retinal scans. The retinal scan is processed using a CNN corresponding to the imaging modality used for acquisition of the retinal scan.

At S30, the computer produces ensembled probabilities of the presence of each of the eye pathoses within the retinal scan.

The ensemble network used in the method of retinal scan image classification comprises at least one neural network for each of the imaging modalities used to acquire retinal scans intended to be processed through the ensemble network. For instance, if there are two distinct imaging modalities used, say, FAF and SD-OCT, there exists at least one neural network intended to handle FAF scans and at least one neural network intended to handle SD-OCT scans. Naturally, multiple neural networks for each imaging modality may be used.

As an example, consider an ensemble network comprising fifteen constituent neural networks. In this example, each neural network is an Inception-v3 deep CNN (CNN), which may take one or more retinal scans of three different imaging modalities from a given patient. Each constituent neural network in this example outputs a gene-level prediction score for 36 individual IRD genes. In this example, these 36 genes are chosen as, collectively, these genes cover over 80% of IRD cases in the European population.

Given a single input retinal scan of one of the three supported modalities, the example ensemble network may apply each of the five networks corresponding to the modality of the scan to obtain a single ensembled image-level prediction. For a set of retinal scans from a single patient, the appropriate group CNN may be applied to each scan in turn. The resulting network- and image-wise predictions may then be combined to produce a single ensembled prediction (or list of probabilities) for the patient by taking an average of the individual (post-softmax) predictions.

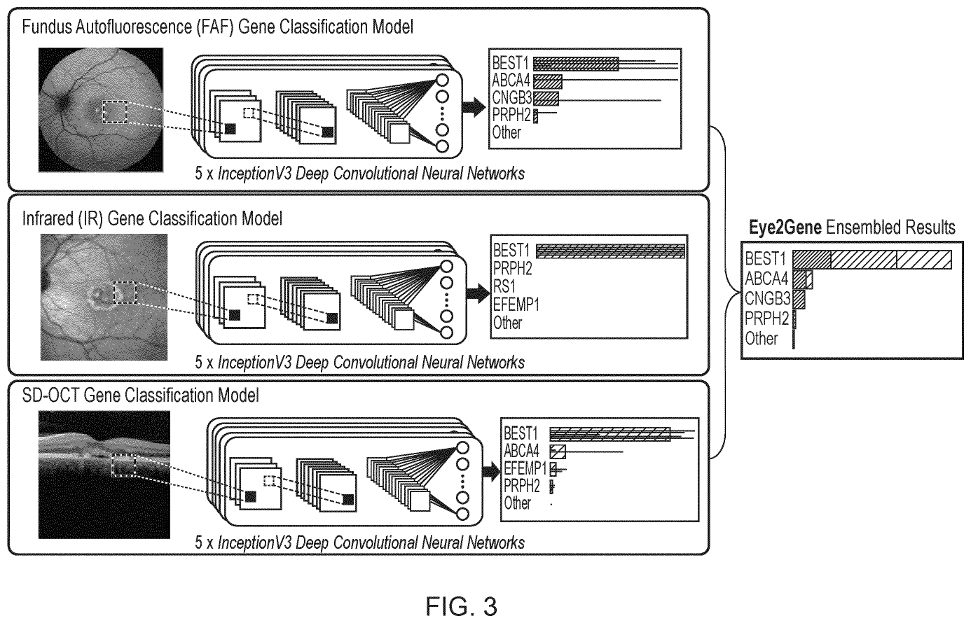

FIG. 3 is an illustrative diagram of the contribution of each of the fifteen constituent CNNs in the above-described example. Each imaging modality-specific predictor block (FAF, IR, and SD-OCT) consists of 5 associated CNNs, configured to provide IRD-gene predictions (that is, prediction of gene abnormalities or mutations) given a retinal scan of the associated imaging modality. Example retinal scans are inset within each predictor block. The CNNs in this example each give a prediction for 36 individual genes. Given a collection of retinal scans from a patient, the appropriate predictor block corresponding to each scan's modality may be applied.

The predictions for all CNNs within each imaging-modality specific predictor block are averaged. For instance, with the illustrated FAF predictor block, 5 separate estimated probabilities of gene abnormalities for BEST1, ABCA4, CNGB3 and PRPH2 (thin bars) are averaged to provide an averaged estimated probability of gene abnormality for each (thick, underlying bar). Only the top 4 genes (in terms of averaged estimated probability) are depicted for each imaging modality. That is, for a retinal scan of a specific modality, a 5-model predictor block may be applied by averaging the output probabilities per gene.

The average estimated probabilities across all scans (from all imaging modalities) in the collection may be taken and used as the final prediction, or ensembled probabilities, for the collection of retinal scans. As seen in the right-most panel (outside the predictor blocks), the ensembled probabilities are obtained by combining the predictions of its fifteen constituent networks using an ensemble approach.

Of course, if retinal scans acquired using only a single imaging modality are processed, the ensembled probabilities will correspond to the averaged estimated probabilities of a single predictor block (for that same single imaging modality).

In the example above, the number of constituent CNNs (fifteen) is merely presented as an example. The skilled reader will appreciate other numbers may also be used. Fifteen is chosen in the example to enable 5-fold cross validation (see below, in respect of training the ensemble network); in k-fold cross validation, the value for k may be chosen such that each training and testing group of data samples is large enough to be statistically representative of the broader dataset. In the present case, k=5 was found to be suitable.

### Training

Any known training techniques suitable for training image classification models may be applied to the constituent CNNs of embodiments, provided that each resultant trained neural network is capable of retinal scan image classification.

In this case, all training code was written in Python with the Keras library, using the TensorFlow backend. Images were loaded into the model training routine via the in-build Keras data-loader, using the Inception-v3 pre-processing function. This loads the images as RGB images, resizes them to the correct input dimensions (256×256 pixels) and rescales pixel values to be between −1 and 1 (by dividing by 127.5, and subtracting 1). Despite the input scans in this example training process being monochromatic, the inventors load them with three colour channels (all set to the same values) for convenience.

For loading images during the training phase, images were also automatically augmented by applying random transformations. [transformations used]

For the network architecture, the inventors used the in-build Inception-v3 model in Keras, using saved ImageNet weights, and stripped the final output layer and included a drop-out layer (ultimately, not used in the final experiments in this example; see below) and a final output layer with 36 outputs and softmax normalisation.

The network was trained in the standard way, using the Adam optimiser on the weighted cross entropy loss of the predicted gene classes versus the true gene. This was done using the Keras fit function, with logging and monitoring via TensorBoard. Network weights were saved at the end of training (after 100 epochs), and the inventors also saved a network config file with the other network settings for record-keeping and easy loading of the network in future via a wrapper class. Classes were reweighted inversely proportionally to how frequently images of each class appeared in the train dataset.

Training in this example was done using an Nvidia Quadro P6000 GPU implemented on a Dell Desktop. Other GPUs, such as Nvidia TITAN Xp, are also suitable. Moreover, conventional CPUs are also able to perform training; CPUs are generally well-suited for smaller training tasks.

As mentioned above, k-fold cross validation is a suitable framework for training the constituent CNNs of an ensemble network. Training data (that is, data used to train the CNNs) contains at least retinal scans and an indication of the pathoses present in the retina of each scan (for instance, an indication of genetic abnormality).

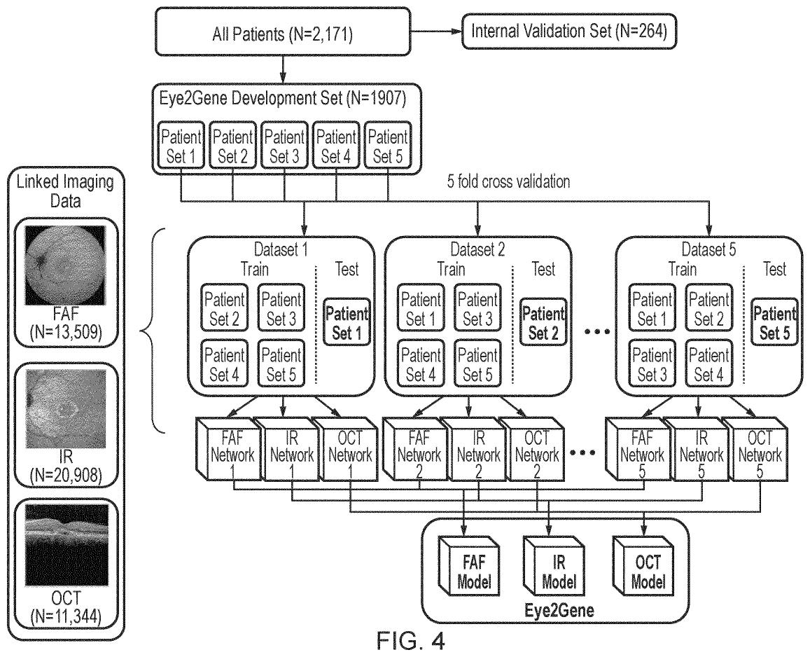

FIG. 4 is an illustrative diagram of k-fold cross validation for training an ensemble network comprising 5 CNNs for three imaging modalities (that is, 5-fold cross validation, giving rise to 15 CNNs).

The above-described training process was repeated in this example for each of the folds for each of the modalities, saving the trained network weights at the end of each run (so early stopping was not used), giving a total of 15 sets of network weights.

The patients in a development set were split into 5 approximately equal sized folds as follows:
1. A table of counts for each fold-modality-gene combination was prepared and initialised to zero.
2. For each patient, the gene and the number of images for each modality pertaining to the patient were ascertained.
3. Patients were randomly shuffled using a set seed (to ensure repeatability).
4. For each patient in turn, the fold with the lowest count across all modalities for the patient's gene in the table was selected, and the patient was assigned to that fold. The counts in the table were incremented by the number of images for that patient for each modality.

For ease of training and evaluating our individual networks, the inventors split the dataset (in CSV format) into a series of train and test CSVs based on the individual folds (so for each modality, there exists files [ . . . ]_train_0.csv, [ . . . ]_test_0.csv, [ . . . ]_train_1.csv, [ . . . ]_test_1.csv, and so on) where [ . . . ]_train_N.csv included folds 0-4, excluding N, and [ . . . ]_test_N.csv included folds N and −1.

To ensure a reasonable amount of train data for each gene, the inventors excluded any genes that did not have at least 5 images across all folds/modalities and excluded all images/patients pertaining to those genes from train and test sets. This gave a list of 36 genes, which were used throughout all experiments.

In the illustrated example, training is performed using retinal scans acquired from patients who have some form of IRD from Moorfields Eye Hospital (MEH). These retinal scans are acquired in patients with IRDs seen at MEH who had undergone genetic testing and where a confirmed genetic cause had been identified by an accredited diagnostic laboratory.

The MEH IRD cohort was previously described by Pontikos, N. et al. (“Genetic basis of inherited retinal disease in a molecularly characterised cohort of over 3000 families from the United Kingdom”. Ophthalmology (2020).) and encompasses 4236 individuals with IRDs caused by variants in 135 distinct genes, of which 452 individuals (with variants in 66 genes) were younger than 18 years of age as of 2019 Aug. 2. Patients with an IRD and a confirmed genetic diagnosis by an accredited genetic diagnosis laboratory were identified, and information about the genetic diagnosis, the age at presentation and mode of inheritance was exported from the MEH electronic health record (OpenEyes) using a SQL query on the Microsoft SQL Server hospital data warehouse database.

Images were exported from the MEH Heidelberg Imaging (Heyex) database (Heidelberg Engineering, Heidelberg, Germany) for all patients with an IRD, based on their hospital number, for records between 2006 Jun. 5 and 2018 Apr. 5. This resulted in a dataset of 1,196,038 images from 2871 IRD patients with disease-causing variants in 132 distinct genes.

TABLE 1
Number of patients and images per gene and modality (FAF, IR and SD-
OCT) for the 36 genes included in the example development dataset.

| | | | | | | | Patients with | Age at first |
|---|---|---|---|---|---|---|---|---|
| | FAF | | IR | | SD-OCT | | images from all 3 | presentation |
| Gene | Patients | Scans | Patients | Scans | Patients | Scans | modalities | range |
| ABCA4 | 521 | 4309 | 575 | 5708 | 535 | 3527 | 472 | 6-81 |
| USH2A | 164 | 1525 | 186 | 1754 | 162 | 1175 | 142 | 9-80 |
| PRPH2 | 118 | 907 | 133 | 1201 | 127 | 850 | 109 | 16-75 |
| RPGR | 111 | 1002 | 133 | 1463 | 120 | 907 | 98 | 4-79 |
| BEST1 | 89 | 990 | 106 | 1291 | 101 | 763 | 81 | 3-80 |
| CHM | 79 | 786 | 87 | 972 | 83 | 599 | 75 | 5-71 |
| RS1 | 71 | 718 | 88 | 1097 | 81 | 567 | 69 | 4-71 |
| RP1 | 69 | 587 | 68 | 574 | 64 | 459 | 62 | 11-78 |
| RHO | 58 | 444 | 70 | 426 | 62 | 311 | 49 | 7-89 |
| PRPF31 | 38 | 362 | 43 | 337 | 36 | 248 | 32 | 7-73 |
| MYO7A | 35 | 326 | 37 | 342 | 31 | 219 | 29 | 5-73 |
| CRB1 | 30 | 328 | 34 | 454 | 25 | 202 | 29 | 7-57 |
| EYS | 29 | 290 | 30 | 298 | 29 | 203 | 25 | 5-69 |
| CNGB3 | 29 | 224 | 63 | 1829 | 54 | 602 | 24 | 3-74 |
| PROM1 | 28 | 204 | 30 | 368 | 24 | 169 | 23 | 7-72 |
| EFEMP1 | 26 | 172 | 30 | 382 | 23 | 147 | 18 | 21-70 |
| TIMP3 | 22 | 153 | 21 | 246 | 17 | 131 | 17 | 3-46 |
| RDH12 | 21 | 169 | 28 | 589 | 20 | 131 | 17 | 36-66 |
| CNGA3 | 19 | 133 | 40 | 843 | 31 | 285 | 16 | 3-55 |
| CACNA1F | 17 | 133 | 23 | 239 | 16 | 85 | 15 | 13-62 |
| RP2 | 17 | 125 | 20 | 204 | 17 | 104 | 15 | 5-71 |
| GUCY2D | 17 | 115 | 22 | 323 | 18 | 109 | 14 | 11-69 |
| RPE65 | 17 | 93 | 31 | 510 | 26 | 158 | 14 | 4-74 |
| BBS1 | 16 | 183 | 18 | 238 | 16 | 127 | 13 | 13-77 |
| NR2E3 | 14 | 216 | 15 | 168 | 14 | 99 | 13 | 25-71 |
| MERTK | 14 | 163 | 14 | 181 | 12 | 90 | 13 | 7-70 |
| CRX | 14 | 113 | 17 | 242 | 14 | 95 | 12 | 3-73 |
| CERKL | 13 | 137 | 13 | 162 | 9 | 71 | 12 | 5-52 |
| MTTL1 | 13 | 71 | 14 | 67 | 14 | 57 | 12 | 6-60 |
| OPA1 | 13 | 64 | 40 | 377 | 35 | 118 | 11 | 5-62 |
| PDE6B | 11 | 157 | 13 | 140 | 12 | 106 | 10 | 26-65 |
| RP1L1 | 11 | 82 | 14 | 184 | 12 | 86 | 9 | 7-57 |
| CYP4V2 | 11 | 80 | 13 | 103 | 10 | 71 | 9 | 18-67 |
| PRPF8 | 10 | 111 | 11 | 99 | 10 | 82 | 8 | 4-52 |
| CDH23 | 9 | 175 | 10 | 92 | 9 | 76 | 8 | 14-67 |
| KCNV2 | 8 | 45 | 13 | 137 | 13 | 70 | 8 | 6-76 |

Note that the full dataset in this example was subject to quality control (see section below). In this way, the dataset may be restricted to only genes with at least 5 images per gene in each of the three modalities (after quality control), leaving 36 individual genes. The distribution of the selected 36 genes is presented in TABLE 1 above. For all 132 genes, a number had insufficient numbers of patients with enough images to properly allocate at least 5 images to each fold and so were excluded.

Note that the full dataset acquired from MEH in this example includes (following data quality control; see below) 15,692 FAF scans, 23,631 IR scans, and 13,099 SD-OCT scans from 2171 patients in 36 distinct genes. This full dataset is split into a ‘development’ set of 1907 patients and a held-out internal test set of 264 patients. The internal test set allows testing of the final ensemble network. The development set allows investigation of network-level properties and ensembling approaches.

As seen in FIG. 4, training in this example is performed using a total of 44,817 retinal scans acquired from 1,907 patients who have some form of IRD (3749 eyes, 6397 appointments) from MEH. These scans are split into the three different modalities: FAF (N=13,509), IR (N=20,098) and SD-OCT (N=11,344). For each of the three modalities, five different neural networks are trained using 5-fold cross validation on the development data resulting in a total of fifteen distinct neural networks

Patients in the development set are divided into 5 approximately equal-sized patient subsets, taking care to ensure an approximately even split across each of the different genes in each of the three different modalities. For each of the three modalities, images from each of the 5 patient subsets are used as the folds in a 5-fold cross-validation set-up. On each combination of four patient subsets, a deep CNN is trained, so each patient subset is withheld from the training set of exactly one network (enabling the images from that subset to be used as test data for that network). As well as diversifying the constituent CNNs of the ensembles, this also enables one to look at the variation in the network-level model (the ensemble network) due to different train/test sets, which can give an idea to how sensitive the ensemble network is to the training dataset.

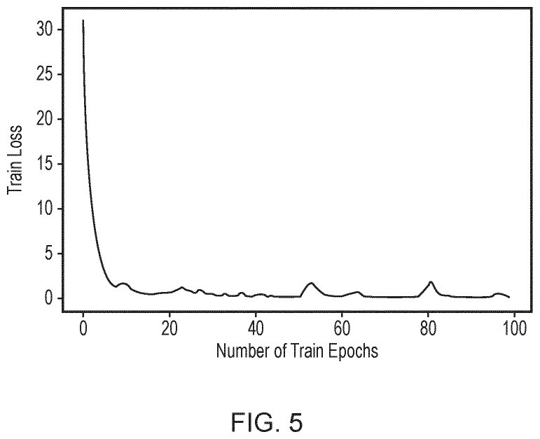

For each of the 15 datasets (three modalities, 5 folds per modality), a 36-class CNN is trained for 100 epochs (passes over the entire dataset), which is found to be sufficient. FIG. 5 demonstrates example network loss on a training set during training. As illustrated, 100 epochs are found to be sufficient for training to converge for all hyper-parameter settings in preliminary investigations on all images from this example dataset.

To obtain values for the various hyper-parameters for training each CNN, for each of the three modalities, one may split the first dataset into further training and validation datasets according to a 75:25 split (giving an effective overall 60:20:20 train/validation/test split). Networks may then be trained on these new training sets using a variety of random hyper-parameter settings; the train-accuracy and average per-gene AUC score (definition below) on the validation set for 10 random hyperparameter settings are recorded in TABLE 2 below.

As seen above, drop-out in the penultimate layer of the network may be used, with various drop probabilities. No dropout is found to work well across all three modalities, hence a dropout probability of 0% is used throughout this example, meaning dropout was effectively absent from the final CNNs.

For cross-validation, in theory, hyperparameter tuning should ideally be applied across each fold independently to avoid biasing to final test results. However, in practice, this significantly increases the compute requirements, while the benefits are likely to be negligible. Thus, the optimal hyperparameter settings are used in this example for the training of the remaining folds as well. This effect only applies to results on the internal development set here and does not affect any of the results on the internal or external test sets.

For each image, the corresponding class label is given by the gene diagnosis of the underlying patient. For the neural networks, an Inception-v3 architecture initialised with pre-trained ImageNet weights is used, where the final output layer is replaced by a linear layer with 36 outputs followed by softmax normalization. The Inception-v3 architecture is chosen as it is the smallest model size amongst a selection of similarly performing architectures applied in other ophthalmology contexts. The skilled reader will appreciate that any other architecture suitable for image classification would also be suitable. The loss function selected in this example is cross entropy loss with class-weighting inversely proportional to gene frequency in the dataset. For the Adam optimizer, the default parameters used in the Keras library (β₁=0.9, β₂=0.999) are selected.

To summarise, for this example, hyperparameter tuning is conducted via random sampling over 10 trials on an 80/20 train/validation split on the first training fold. Following hyperparameter tuning, a batch size of 128, learning rate of 0.0001 and dropout probability of 0% are found to be optimal. These hyper-parameters are used for the training of the networks across all three modalities. To avoid overfitting on the training data, data augmentation techniques may also be applied (see section below).

Returning to FIG. 4, 5-fold cross validation trains CNNs for each imaging modality used during acquisition of the dataset.

### Training Data Quality Control & Augmentation

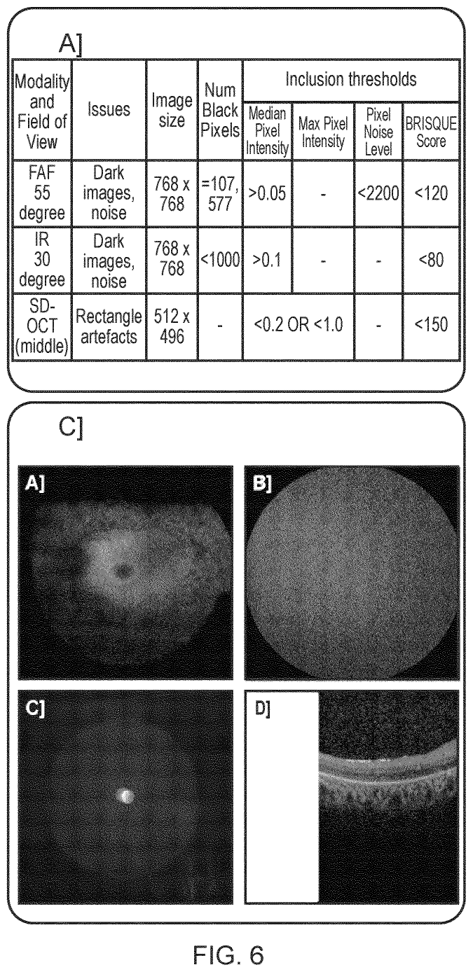

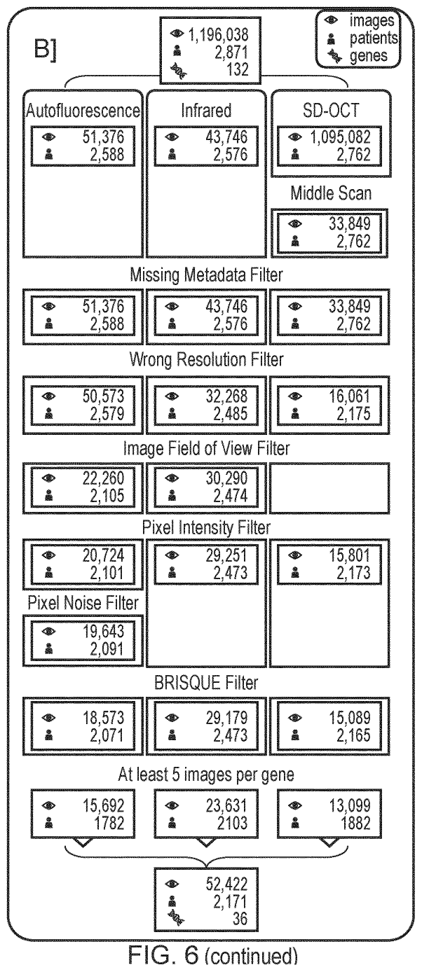

Prior to training, the dataset in hand may be subject to pre-processing to provide quality control. Note that this step is not always necessary and is dependent on the dataset in question. In the above-described dataset (from the Heyex database of MEH), quality control is implemented. FIG. 6 provides a schematic overview of an example quality control process. The table in panel A provides quality control steps and inclusion thresholds applied for the three imaging modalities in the above-described example.

Of the database query, retinal scans are divided by modality, with 51,376 FAF scans, 43,746 IR scans, and 1,095,082 SD-OCT scans (leaving 5,834 images of other modalities). Since SD-OCT produces multiple B-scans, for each B-scan only the image corresponding to the central B-scan that traverses the fovea is used, as it is likely to be the most informative B-scan. Following this, 33,849 OCT B-scans remain.

For all three modalities, filtering is applied. Any corrupted scans, and any scans in sizes other than 768×768 pixels (for FAF and IR scans) or 512×496 (for OCT scans) are discarded. Additionally or alternatively, scans may be resized. Both the IR and FAF scans feature two different imaging magnification levels, 30 degrees and 55 degrees. In each case, only the most common mode (55 degrees for FAF, and 30 degrees for IR) is kept, and all other scans are discarded. These two modes may be distinguished automatically by checking the number of black background pixels (RGB 0,0,0), where a total of 107,577 black pixels is found to correspond to 55 degrees images.

To remove low-quality and defective images a number of quantitative filters are applied. The types of filters may be determined by initial inspection of the data to identify any particular issues. Thresholds for these filters are set by examining a sample of scans rejected by the filter and adjusting the threshold until over 25% of rejected scans are judged to be of a reasonable quality level.

In particular, FAF and IR datasets both contain a number of excessively dark scans. Accordingly, scans with a median pixel intensity below 0.05 for FAF, and 0.1 for IR are rejected.

Additionally, many FAF scans featured a large amount of noise. To measure this noise level, the inventors introduce a ‘Pixel Noise Level’ score, where the original scan is compared to a blurred version (using a normalized box filter with a 5×5 kernel) of the same image, taking the sum of the squared difference between the two images. FAF scans with a total squared difference of over 2200 are rejected. Many OCT images contained artifacts consisting of large regions with a pixel value of 1.0 (i.e., the maximum, in this case). To remove these, any images with a maximum pixel value of 1.0, and also with median intensity greater than 0.2 are rejected.

Finally, a BRISQUE score (Blind/Referenceless Image Spatial Quality Evaluator, a no-reference image quality score) is computed for each scan, using the PyBRISQUE library, and scans above a certain threshold are discarded. This threshold in this example is set at 120 for FAF images, 80 for IR images, and 150 for OCT images.

Following quality control, and following restriction of the dataset to only genes that have at least 5 scans, 15,692 FAF, 23,631 IR and 13,099 OCT scans remain in 36 most common genes, as seen at the bottom of panel B.

The skilled reader will appreciate that the specific pre-processing quality control procedures above are dataset dependent, and the exact parameters will depend on the dataset at hand.

Panel C provides examples of poor-quality scans rejected by the above-described example filters. Subpanel A is a FAF scan in which it is too dark to make out any details. Subpanel B is a FAF scan with excessive amount of noise. Subpanel C is an Infra-red scan in which it is too dark to make out any details. Subpanel D is an SD-OCT scan with a large artefact obscuring part of the scan.

As mentioned above, data augmentation may be used to avoid overfitting on training data. Retinal scans may be augmented prior to training such that the augmented training dataset comprises the same number of retinal scans as the original training dataset, or such that augmented retinal scans are concatenated to the original training dataset.

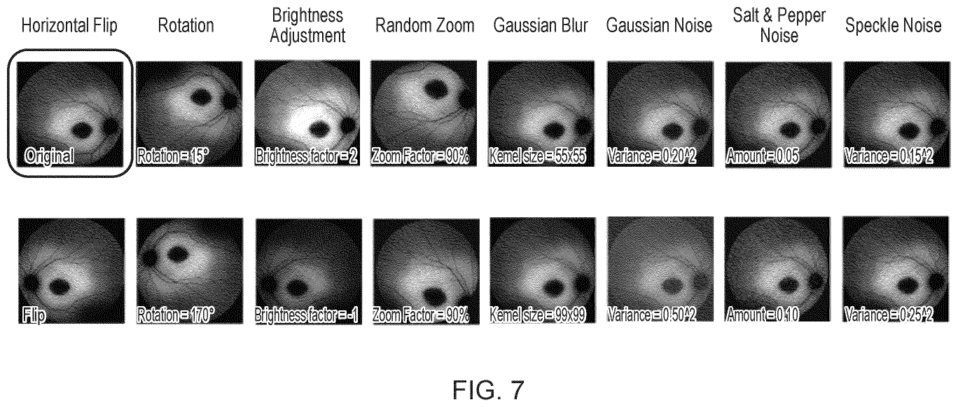

FIG. 7 demonstrates 8 data augmentation techniques that may be applied to retinal scans. Depicted techniques include horizontal flipping; rotation; brightness adjustment; random zoom; Gaussian blurring; addition of Gaussian noise; addition of Salt & Pepper noise; and addition of Speckle noise. As seen from the bottom row of images, techniques may be applied in series; for example, the original image is horizontally flipped and a brightness factor of −1 is applied, resulting in the image third in from the left. Any or all of the data augmentation techniques may be applied to retinal scans acquired from any imaging modality.

### Performance Evaluation—Efficacy on Example Cross-Validation Test Sets & Held-Out Validation Test Set

As in the above example, the constituent neural networks of the ensemble network may be trained via a 5-fold cross-validation approach where each individual network is trained on a distinct subset of training data.

For the above example, for the networks trained on FAF images, the inventors observe an average top-1 accuracy across networks of 44.3% (CI_{95%}=42.5-46.2), top-5 accuracy of 72.7% (71.0-74.4), and average per-gene ROC AUC of 0.827 (0.812-0.842). For the networks trained on IR images, the inventors observe an accuracy of 45.8% (43.6-47.9), top-5 accuracy of 73.7% (72.2-75.2), and AUC of 0.834 (0.824-0.844). Finally, for the networks trained on SD-OCT images, the inventors observe an accuracy of 51.6% (48.4-54.9), top-5 accuracy of 77.4% (75.2-79.6), and AUC of 0.845 (0.831-0.859).

The results on the held-out patient set are similar, with a mean accuracy across models of 51.4% for FAF, 46.9% for IR, and 56.3% for SD-OCT models (see TABLE 4 below; in particular the single model average, single image section (top-left)).

In more detail, to evaluate the efficacy of the ensemble network approach, the inventors simulate a scenario of applying the approach to scans taken during a patient visit by applying the approach to scans from the internal held-out dataset (validation set), introduced above (see FIG. 4). The Moorfields held-out dataset is used for internal testing. As described above, the MEH internal test dataset in this example consists of 1900 scans from 264 patients across 32 gene diagnoses. The breakdown of scans per modality is given in the first row of TABLE 3 below.

TABLE 3
Number of patients, scans, and genes in
internal and external test datasets.

| | Number | Number of Images | | | | Number of |
|---|---|---|---|---|---|---|
| Site | Patients | FAF | IR | SD-OCT | Total | Genes |
| Moorfields | 264 | 733 | 692 | 475 | 1900 | 32 |
| Oxford | 59 | 106 | 120 | 120 | 346 | 29 |
| Liverpool | 35 | 70 | 70 | 70 | 210 | 15 |
| Bonn | 127 | 241 | 0 | 232 | 473 | 11 |
| São Paulo | 15 | 16 | 36 | 52 | 104 | 3 |
| Total | 500 | 1166 | 918 | 949 | 3033 | 36 |

Scans in the internal test dataset are grouped by patient and by date of acquisition. Predictions are compared to the underlying genetic diagnoses of the respective patients. Using the scans from the first appointment only for each patient, the ensemble network approach attains a top-1 accuracy of 66.7%, top-5 accuracy of 85.6%, and an average per-gene receiver operator characteristic AUC of 0.935 (see TABLE 4; 5-model ensemble, multiple images/modalities section (bottom-right)).

The ensemble network approach is applied across all available scans per patient per appointment (defined as all scans from a given patient at a given date), and on a per-patient level (using all the available scans per patient across all appointments).

TABLE 4
Overview of test accuracies on held-out patient sets using different methods
of combining model predictions. Per visit means all eye scans for each patient
visit. Per patient means all eye scans across multiple patient visits.

| | | Single image | | | Multiple images/modalities | | |
|---|---|---|---|---|---|---|---|
| | | FAF | IR | SD-OCT | First Visit | Per Visit | Per Patient |
| Single model | Acc | 51.4% | 46.9% | 56.3% | 62.9% | 62.8% | 64.9% |
| average | Top-2 | 63.9% | 59.2% | 67.3% | 71.7% | 72.3% | 74.1% |
| | Top-3 | 70.9% | 66.8% | 73.3% | 78.5% | 77.8% | 80.3% |
| | Top-5 | 77.8% | 76.6% | 81.4% | 82.9% | 84.6% | 86.5% |
| | Top-10 | 87.1% | 87.8% | 89.3% | 91.3% | 92.1% | 93.8% |
| | Mean AUC | 0.835 | 0.843 | 0.876 | 0.912 | 0.914 | 0.927 |
| 5-model | Acc | 59.2% | 55.3% | 61.5% | 66.7% | 68.1% | 66.7% |
| Ensemble | Top-2 | 69.2% | 64.4% | 72.0% | 75.4% | 76.8% | 78.4% |
| | Top-3 | 74.5% | 71.2% | 76.8% | 80.7% | 82.1% | 83.3% |
| | Top-5 | 81.3% | 80.8% | 82.9% | 85.6% | 88.1% | 89.4% |
| | Top-10 | 90.2% | 91.2% | 91.9% | 94.7% | 94.2% | 95.1% |
| | Mean AUC | 0.881 | 0.885 | 0.911 | 0.935 | 0.937 | 0.950 |

For each modality-specific ensemble model, the model is applied to all scans of that modality from the internal test set. The model predictions are then compared against the underlying gene diagnoses for each patient to compute the overall accuracy of the model on the test data, the top-k accuracy (the proportion of images where the correct gene is within the highest k predictions of the network) for k=2,3,5, 10, and the average per-class Area Under the Receiver Operator Characteristic curve (AUROC).

The ROC (receiver operator characteristic) demonstrates the trade-off between specificity (false positive rate) and sensitivity (true positive rate) for a given gene. For each gene, the AUC (Area Under the Curve) of the corresponding ROC curve for the model is obtained by comparing the model predictions for the given gene in a one-versus-all setup.

Confidence intervals for accuracies and AUCs on the development data are obtained by taking the standard deviations of the accuracy/AUC for each of the 3 modalities across the 5 networks and estimating a 95% confidence interval via the expression.

Performance Evaluation—Ensemble Approach v. Others

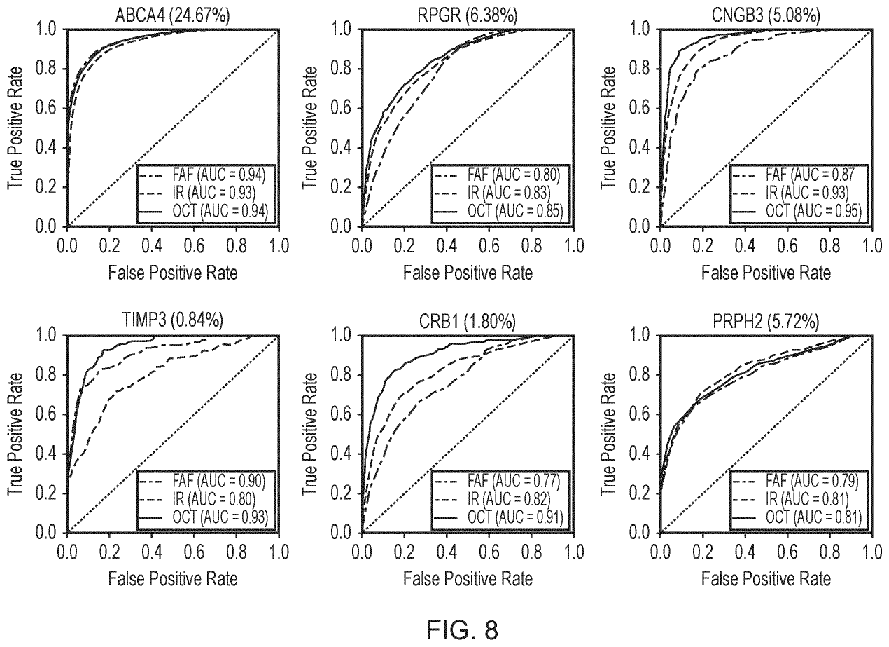

By breaking down the ensemble network into its constituent networks, it is possible to see the overall accuracy across each modality is relatively similar. FIG. 8 is a collection of per-gene Receiver Operating Characteristic (ROC) curves from cross-validation data for the three different imaging modalities across 6 of the 36 predicted genes in the above-described example. FAF classification is depicted with dotted-dashed curves; IR classification is depicted with dashed curves; OCT classification is depicted with solid curves. Each curve takes the mean ROC curve across the 5 models per modality. Percentages next to gene name denote the percentage of total images (across all three modalities) of the given gene. Shaded regions denote regions within one standard error of the mean. Only 6 genes (ABCA4, RPGR, CNGB3, TIMP3, CRB1, and PRPH2) from the 36 classified genes in this example are depicted here, for simplicity.

It is also possible to see that it is possible to see that, while the overall accuracy across each modality is relatively similar, there are some notable differences for certain genes. For example, the FAF and SD-OCT networks are significantly better at detecting TIMP3 than the IR networks. This is notable as TIMP3-retinopathy is associated with an increase in signal in the peripheral macula in FAF imaging and drusen-like deposits in SD-OCT scans. Similarly, the SD-OCT networks are better at detecting CRB1 than the IR and FAF networks. CRB1-retinopathy is often characterized by a thicker retina with less defined layers, something which cannot be distinguished in FAF or IR imaging.

Many genes follow a similar pattern to PRPH2 with little deviation between modalities. However, for some genes, there are pronounced differences in the abilities of the networks of different modalities to distinguish the gene in question. For example, for CRB1, a gene associated with thickening of the retina, SD-OCT is the most predictive modality.

Thus, an ensembled approach to retinal scan image classification is able to identify optimal image modalities on a per-pathoses level (e.g., on a per-gene level).

It is found that combining predictions across multiple models (ensembling) and across multiple images acquired during one or more patient visits contributed to the performance of ensemble networks.

For applying ensembling of the five models per modality to the individual images, the inventors observe accuracies of 59.2%/55.3%/61.5% for FAF/IR/OCT, top-5 accuracies of 81.3%/80.8%/82.9%, and AUCs of 0.881/0.885/0.911 (see TABLE 4; 5-model ensemble, single image section (bottom-left)). This approach yields improved results relative to the single model average, single image approach.

For combining individual model predictions across multiple images (without ensembling the 5 models per modality) at the per-visit level, on the held-out test set in the above-described example, the inventors see an overall mean accuracy across models of 62.8%, top-5 accuracy of 84.6%, and mean AUC of 0.914 (see TABLE 4; single model ensemble, multiple images/modalities section (top-right), per visit column).

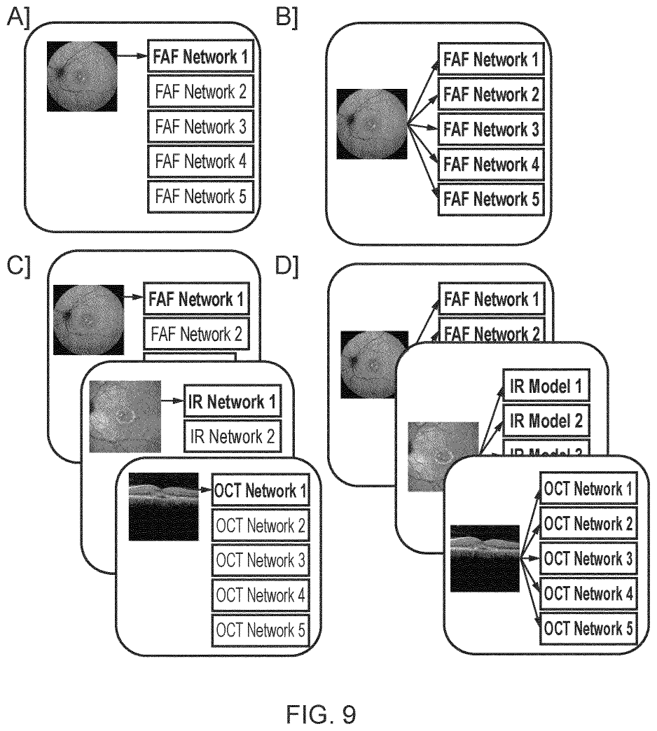

FIG. 9 is a schematic overview of the four types of models benchmarked here. Panel A illustrates a single-image model, which takes a single image from one eye as input and outputs a single classification probability from a single network. Panel B illustrates the single-image ensemble model (five networks for each modality), which takes a single image from one eye as input and outputs classification probabilities from each of the five networks in the ensemble, which are combined into a single classification probability. Panel C illustrates the multi-image model (of a single network for each modality), which takes multiple images of different modalities (i.e., FAF, OCT and IR) as input and outputs a classification probability for each image, which are combined into a single classification probability for the patient. Panel D illustrates the multi-image ensemble (of five networks for each modality), which takes multiple images of different modalities (i.e., FAF, OCT and IR) as input and output classification probabilities from each of the five networks for each image, which are combined into a single classification probability for the patient.

TABLE 5 below summarises the accuracy when classifying multiple scans using a single convolutional neural network for each imaging modality in comparison to the accuracy when classifying multiple scans using a 5-model per imaging modality ensemble network. Note that the single image accuracy results (left column) are the same as the single image accuracy results provided in TABLE 4 (left column) and are included here for ease of reference.

TABLE 5
Overview of test accuracies on held-out patient sets using single scan v. multiple scans.

| | | Single image | | | Multiple scans | | | |
|---|---|---|---|---|---|---|---|---|
| | | | | SD- | | | SD-OCT | |
| | | FAF | IR | OCT | FAF only | IR only | only | All three |
| Single model | Acc | 51.4% | 46.9% | 56.3% | — | — | — | 62.9% |
| average | | | | | | | | |
| 5-model | Acc | 59.2% | 55.3% | 61.5% | 59.1% | 62.1% | 62.9% | 66.7% |
| Ensemble | | | | | | | | |

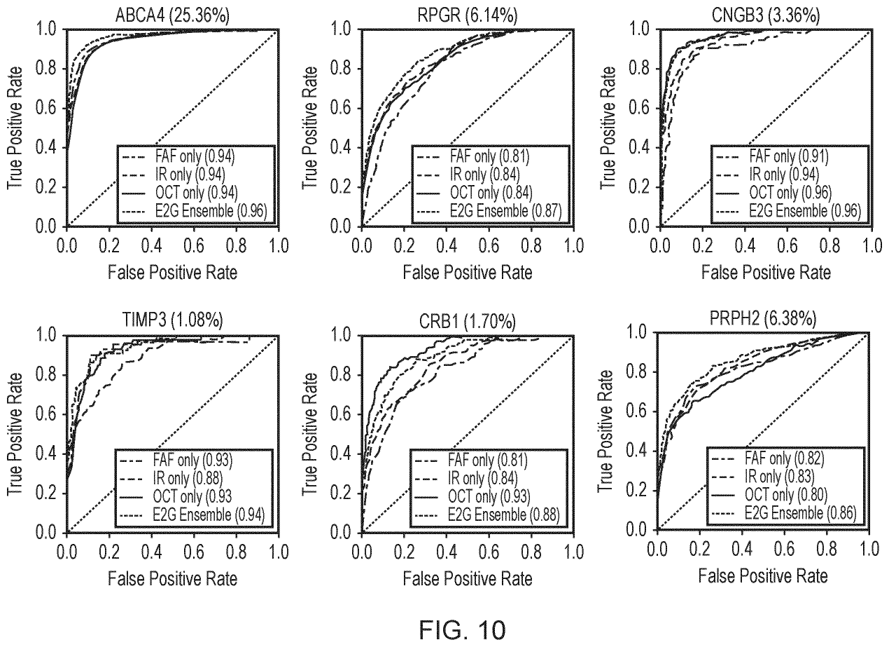

The inventors find that combining all images across all modalities outperform the best performing modality (i.e., restricting to images of that modality only) on the majority of genes, based on the per-gene ROC AUC, demonstrating the advantage of the multi-modality approach. FIG. 10 is a collection of per-gene ROC curves from cross-validation data for the combined prediction on all images from a given patient appointment (solid black curves; “E2G Ensemble”), overlaid on the multi-image ROC curves when restricted to only images of a given modality. Percentages next to gene names denote the percentage of total appointments corresponding to the given gene. For brevity, only 6 ROC curves of the 36 for all gene classifications in this example are provided (the same 6 as in FIG. 8). Again, this demonstrates the advantage of the multi-modality approach.

With both ensembling multiple models per modality applied to individual images, and combining individual model predictions across multiple images, these cases offer results superior to single-network results but are inferior to the overall ensemble model approach taken in embodiments, indicating that both employing ensembling, and combining predictions across multiple images is advantageous.

### Performance Evaluation—Robustness

To ensure results are generalisable across hospitals in respect of the above-described example case, the inventors applied the classifier to data from four IRD clinics which included, Oxford Eye Hospital (UK), Liverpool University Hospital (UK), University Hospital Bonn (Germany), and the Federal University of São Paulo (Brazil). As indicated above in TABLE 3, Oxford Eye Hospital (UK) provided a sample of 346 scans from 59 patients with distinct gene diagnoses in 29 different genes. The University Eye Hospital of Liverpool (UK) provided a sample of 210 scans from 35 patients with distinct gene diagnoses in 15 different genes. The University Eye Hospital Bonn (Germany) provided a sample of 473 scans from 127 patients with distinct gene diagnoses in 11 different genes. The Federal University of Sao Paulo (Brazil) provided a sample of 104 scans from 15 patients with distinct gene diagnoses in 3 different genes.

Patients and scans were selected by clinicians based at these different clinics, who were instructed to select patients with a confirmed genetic diagnosis, who had a number of scans available for each modality. A selection of scans was shared with the inventors, along with the genetic diagnosis for each patient, with the exception of data from Bonn University, where they instead ran an embodiment locally, to avoid having to transfer any patient data.

The inventors ran an ensemble network on images from each patient and compared the prediction of the ensemble network to the underlying gene diagnosis. Combining all data from across all four sites (of 1133 retinal scans from 236 patients), an overall accuracy of 65.3%, and a top five accuracy of 86.4% (a breakdown of results at a per-site level is shown in TABLE 6 below) was found.

Since the distribution of genes in the external test data was different to the main MEH dataset (and hence the internal held-out test set), and the accuracy of the ensemble network in this example varies across genes, the inventors also recorded the per-gene prevalence (proportion of the dataset) and sensitivity (accuracy on that particular gene) for each gene at each of the external sites. To understand how much the difference in accuracy on the external data was due to differences in gene distribution, for each site the inventors calculated the reweighted accuracy on the internal test data to match the gene distribution of the target external dataset. Doing this, it is found that, assuming consistent performance gene-for-gene, one would expect to see an overall accuracy of 67.2% on the external data, which is still slightly higher than the actual figure of 65.3%. This pattern was consistent across all four sites, where the actual accuracy was found to be a few percent lower than the internal test data would predict.

To summarise, ensemble networks trained in accordance with embodiments are robust and generalisable across sites when faced with previously unseen datasets.

TABLE 6
Overview of ensemble network results on the external data across
the different sites. Gene-For-Gene Extrapolation from internal
dataset is the sum of the per-gene sensitivity on the MEH
internal test dataset multiplied by the gene frequency of
the external dataset and represents the accuracy one would
expect to see for a gene distribution of that site.

| | | Num. | | Gene-For-Gene |
|---|---|---|---|---|
| | Num. | Unique | | Extrapolation |
| Site | Patients | Genes | Accuracy | from Moorfields |
| Oxford | 59 | 29 | 54.2% | 58.4% |
| Liverpool | 35 | 15 | 57.1% | 59.9% |
| Bonn | 127 | 11 | 70.1% | 70.3% |
| Säo Paulo | 15 | 3 | 86.7% | 92.3% |
| Combined (external | 236 | 30 | 65.3% | 67.2% |
| only) | | | | |
| Moorfields | 264 | 32 | 66.7% | — |
| Combined (all test | 500 | 36 | 66.0% | — |
| data) | | | | |

### Performance Evaluation—Relative to Human Specialists

To contextualize the performance of this example embodiment relative to human specialists, the inventors asked nine ophthalmologists with varying levels of experience to predict the causative gene based on a single FAF image across 50 different patients (chosen to get a uniform representation of all genes in the dataset) from the internal held-out test set in the above-described example. On this task the ophthalmologists achieved an average accuracy of 17%, compared to 38% for the trained ensemble network on the same images. The inventors also asked a further four ophthalmologists to predict the causative genes for a selection of patients from the University Hospital Bonn external test set, asking them to choose from just the 11 genes in the dataset. On this task they achieved an average accuracy of 42%, compared to an accuracy of 49% for the trained ensemble network (where the trained ensemble network still had to choose among 36 genes).

In more detail, the results of the human benchmarking by ophthalmologists are presented in TABLE 7 below. In this task, ophthalmologists were asked to identify the correct diagnostic gene when shown an FAF scan of a patient with an IRD. As expected, human performance tended to improve with level of experience. The inventors also applied the trained example ensemble network to the same datasets, restricted to single-image predictions (so just using the 5 model FAF ensemble) for a fair comparison. The performance of the trained ensemble network was generally better than any single human expert (except for ophthalmologist 11 who obtained 60.3% accuracy) and the frequency that the correct gene appeared in the top 5 predictions of the trained ensemble network was comparable to the frequency it appeared in all human guesses.

Thus, retinal scan image classification using an example trained ensemble network is comparable to experts.

TABLE 7
For comparison, ophthalmologists and a trained ensemble network
according to an embodiment classified 50 FAF retinal scans
of 50 patients results from Moorfields Eye Hospital internal
held-out test dataset and also on Bonn external test datasets
which included 73 FAF retinal scans from 37 patients.

| | | | |
|---|---|---|---|
| IRD | Experience | Correct | Correct |
| spe- | level | top | top-5 |
| cialist | (years) | guess | guesses |

Moorfields human benchmark dataset (N = 50)

| | | | | |
|---|---|---|---|---|
| Ophthalmologist 1 | N | <10 | 6 (12%) | 13 (26%) |
| Ophthalmologist 2 | N | <10 | 6 (12%) | 11 (22%) |
| Ophthalmologist 3 | N | 10-15 | 10 (20%) | 15 (30%) |
| Ophthalmologist 4 | N | <10 | 8 (16%) | 15 (30%) |
| Ophthalmologist 5 | Y | 10-15 | 12 (24%) | 16 (32%) |
| Ophthalmologist 6 | N | <10 | 9 (18%) | 15 (30%) |
| Ophthalmologist 7 | N | <10 | 10 (20%) | 15 (30%) |
| At least one | — | — | 31 (62%) | 39 (78%) |
| ophthalmologist | | | | |
| is correct | | | | |
| Example trained | — | — | 19 (38%) | 35 (70%) |
| ensemble network | | | | |
| (FAF only) | | | | |

Bonn human benchmark external test dataset (N = 73)

| | | | | |
|---|---|---|---|---|
| Ophthalmologist 10 | N | <10 | 25 (34.3%) | 60 (82.2%) |
| Ophthalmologist 11 | Y | 10-15 | 44 (60.3%) | 61 (83.6%) |
| Ophthalmologist 12 | Y | 10-15 | 36 (49.3%) | 57 (78.1%) |
| Ophthalmologist 13 | N | <10 | 25 (34.3%) | 55 (75.3%) |
| At least one | — | — | 54 (74.0%) | 67 (91.8%) |
| ophthalmologist | | | | |
| is correct | | | | |
| Example trained | — | — | 36 (49.3%) | 60 (82.2%) |
| ensemble network | | | | |

Note:
ophthalmologists 8 and 9 did the quiz together.

### Performance Evaluation—Ensemble Network Prediction Calibration

While the example ensemble network outputs predictions in terms of probabilities for each gene, for any given image each underlying network typically predicts confidently for a single gene class, making it difficult to ascertain model confidence from the outputs of any single network alone.

However, the inventors find that that, when the constituent network predictions are combined in the ensemble network, other than a consistent over-prediction by 10%, the output predictions appeared relatively well-calibrated. For example, if the ensemble network predicts a given gene with 70% probability, that prediction was correct approximately 60% of the time.

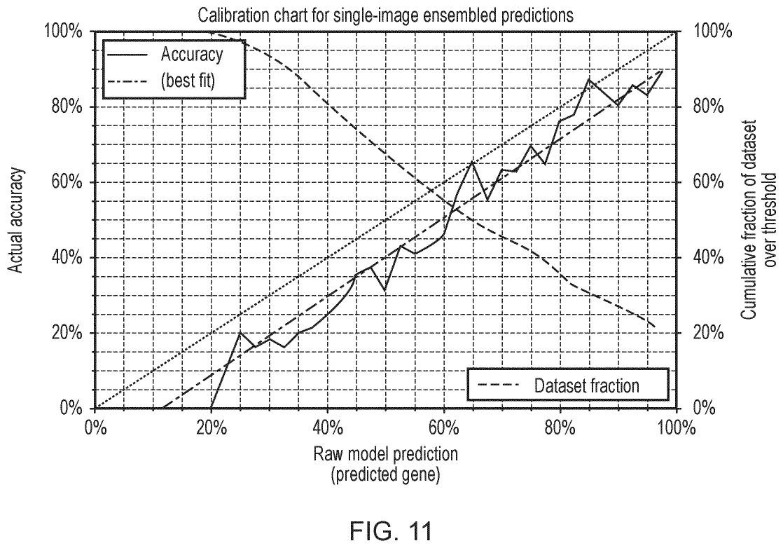

This is shown in FIG. 11, which demonstrates actual accuracy compared to model confidence for an ensemble network on individual images, along with line of best fit (slope=1.04, intercept=−11.2%). The ensemble prediction is slightly overconfident compared to a ‘well-calibrated’ 1:1 correspondence with actual accuracy (shown by dotted line). The total proportion of the dataset above each confidence threshold is also shown (dashed line).

This is useful for providing accurate and informative feedback to the end user as to the confidence of the model. In particular, this aspect may be used in implementations of embodiments in web-apps (see below) to identify unclear input scans which may benefit from being retaken. This may be implemented by flagging the individual images that are below a certain confidence threshold when passed through the model.

### Refining Ensemble Network Output—Inclusion of Age and Mode-of-Inheritance

In addition to retinal scans, further information such as the age at which a patient is presented to the hospital and their family history can be useful in determining the eye pathoses, for instance the causative gene. Since this information is frequently available alongside patient scans, the method of retinal scan image classification using an ensemble network may be modified to incorporate these additional features. In particular, modifications to embodiments enable combining patient age at first presentation (age) and mode of inheritance (MOI), which can be inferred from family history, with the output of ensemble networks model/One manner of incorporating this information is through use of a linear classifier, for example implementing logistic regression without regularisation. By training a linear classifier (using logistic regression without regularisation) to predict the gene class based on the output of the ensemble network, and age and MOI, for the above-described example, it is found that this improves the overall accuracy from 66.7% to 68.9%, top-5 accuracy from 85.9% to 89.4%. In this instance, this did not improve mean per-gene AUROC, which remained unchanged at 0.935. See TABLE 8 below for a detailed breakdown of the efficacy of a combination of approaches.

TABLE 8
Results of various classification approaches on internal test dataset.

| | | | | | |
|---|---|---|---|---|---|
| | | | | Ensemble | Age + Mol + |
| | | | Age + | network | Ensemble |
| Features | Age | Mol | Mol | only | Network |

Per image

| | | | | | |
|---|---|---|---|---|---|
| Acc | 32.0% | 37.4% | 38.6% | 58.2% | 64.7% |
| Top-2 | 41.7% | 52.1% | 55.0% | 67.9% | 73.5% |
| Top-3 | 50.9% | 64.1% | 60.5% | 73.7% | 79.4% |
| Top-5 | 61.1% | 71.8% | 74.0% | 81.5% | 85.6% |
| Top-10 | 83.2% | 85.2% | 84.2% | 91.1% | 93.1% |
| Mean AUC | 0.606 | 0.719 | 0.751 | 0.892 | 0.913 |

First appointment only

| | | | | | |
|---|---|---|---|---|---|
| Acc | 29.5% | 37.1% | 37.5% | 66.7% | 68.9% |
| Top-2 | 44.7% | 54.2% | 53.8% | 75.4% | 80.3% |
| Top-3 | 51.5% | 64.4% | 61.7% | 80.7% | 84.8% |
| Top-5 | 59.1% | 73.1% | 73.5% | 85.6% | 89.4% |
| Top-10 | 78.0% | 83.0% | 85.2% | 94.7% | 94.3% |
| Mean AUC | 0.575 | 0.708 | | 0.935 | 0.935 |

Per appointment

| | | | | | |
|---|---|---|---|---|---|
| Acc | 30.4% | 36.8% | 38.6% | 68.1% | 71.6% |
| Top-2 | 45.2% | 53.7% | 53.7% | 76.9% | 79.0% |
| Top-3 | 51.6% | 62.2% | 60.7% | 82.1% | 82.9% |
| Top-5 | 60.1% | 71.9% | 69.8% | 88.1% | 88.1% |
| Top-10 | 79.6% | 83.7% | 85.5% | 94.2% | 94.3% |
| Mean AUC | 0.578 | 0.692 | 0.726 | 0.937 | 0.934 |

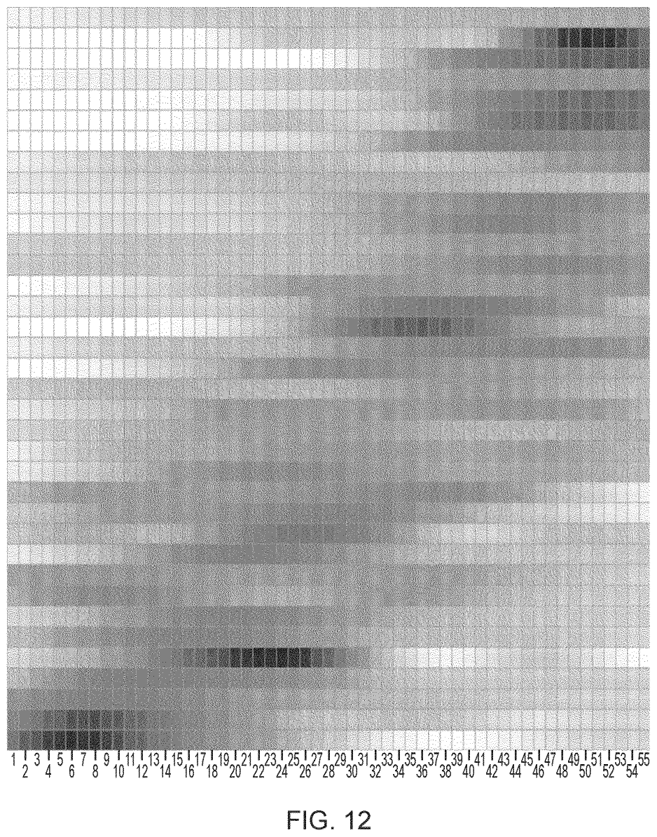

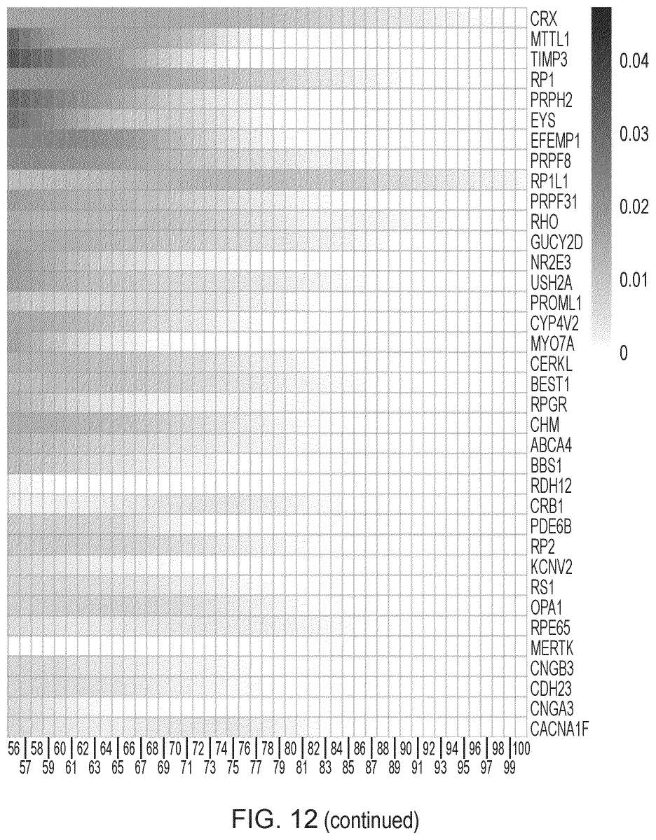

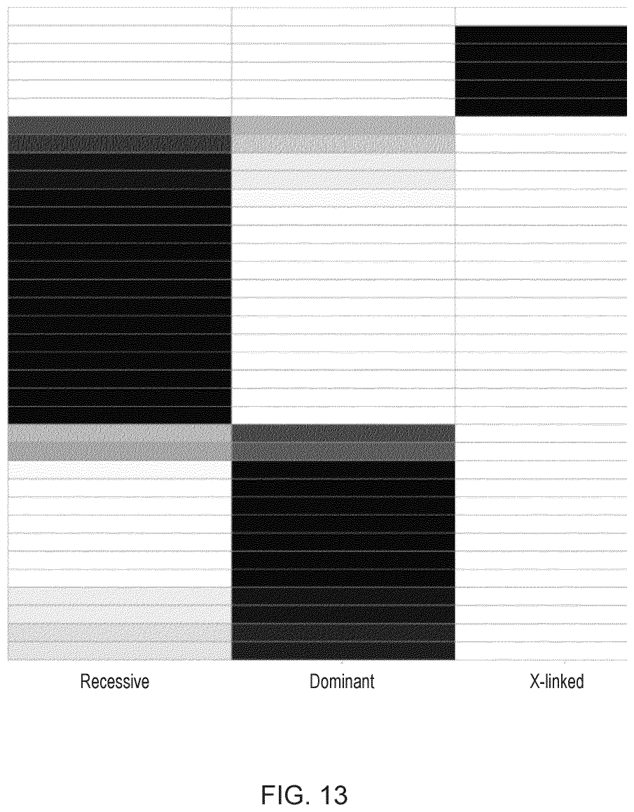

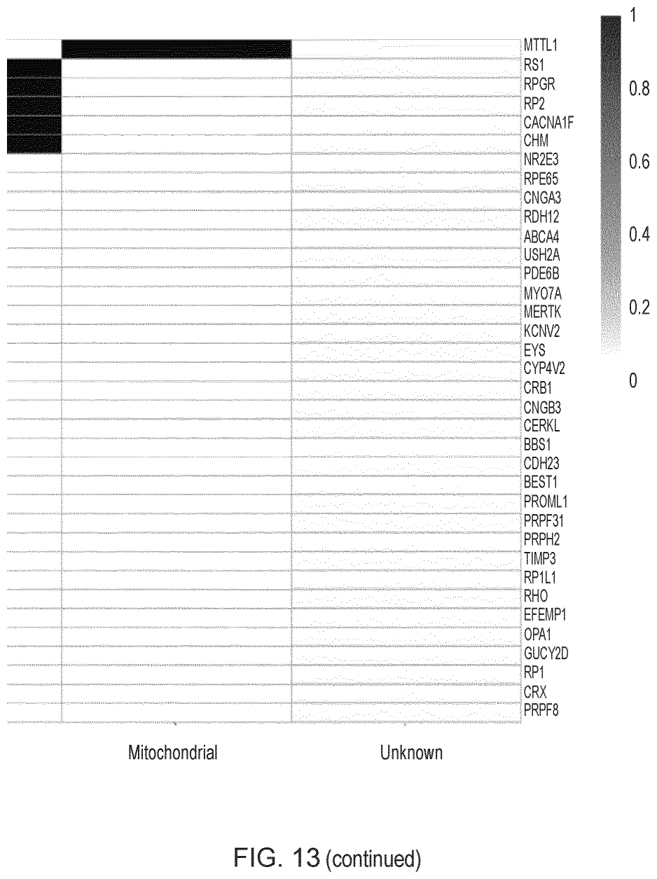

In more detail, age, and mode of inheritance (MOI) are important factors to consider in the genetic diagnosis of IRDs as these provide strong prior evidence towards a specific gene. For example, if the patient is young and the MOI X-linked this may suggest RP2 as an associated gene. FIG. 12 indicates a distribution of age at earliest presentation per genetic diagnosis, for the example described throughout this specification. The horizontal axis provides the age of the subject for which each genetic diagnosis (abnormality of gene, as provided in the vertical axis) first presents. As examples, the age of earliest presentation of abnormalities with the CACNA1F gene is within childhood, with the strongest presence from around 1 to 12 years old; the age of earliest presentation of abnormalities with the MTTL1 gene is later in life, with the strongest presence in the late forties and the early fifties. FIG. 13 indicates known mode of inheritance per gene, for the same example; as an example, it is known that the MOI of abnormalities in the MTTL1 gene is likely mitochondrial based. Note that some genes such as BEST1 and PROML1 can be either dominant or recessive.

To incorporate these non-imaging features, a model stacking approach may be taken, where a linear classifier may be trained on the combined predictions from ensemble network and the additional input features, which then produced a single prediction based on the original model prediction and the additional features.

In this example, age is treated as a normalised numeric variable (subtracting the mean and dividing by standard deviation) and MOI as a one-hot vector with five classes: recessive, dominant, X-linked, mitochondrial, and unknown. For example, recessive genes may be encoded with a 1×5 row vector with values [1 0 0 0 0]. For each image/appointment from the five patient sets in this example, the inventors concatenate these features with the final output of the corresponding network (where each gene prediction is treated as an individual feature) and use these, along with the true gene labels, to fit a logistic regression classifier (without regularization).

These feature vectors concatenated with the predictions of ensemble network from the cross validation experiments (i.e., using just the test results for each network) form 41-wide feature vectors, upon which a 36-class linear classifier is fit using logistic regression (with the target of the patient genes, as before). This trained classifier is applied to the internal test set, using the concatenated age+MOI features and the ensemble network predictions as before, and the outputs are compared to the underlying genes for each patient.

To test the resulting combined model, the inventors apply this trained model to the internal test set, using the ensemble predictions from the full ensemble network concatenated with age and MOI, and using the resulting predictions as the new gene-level predictions, performing the same top-k and mean per-gene AUC analysis as for the other performance evaluations. The inventors also perform a cross-validation analysis based on the five folds in the development set where the model outputs are used on 4 folds as train data, and tested on the remaining fold, which is repeated for each fold.

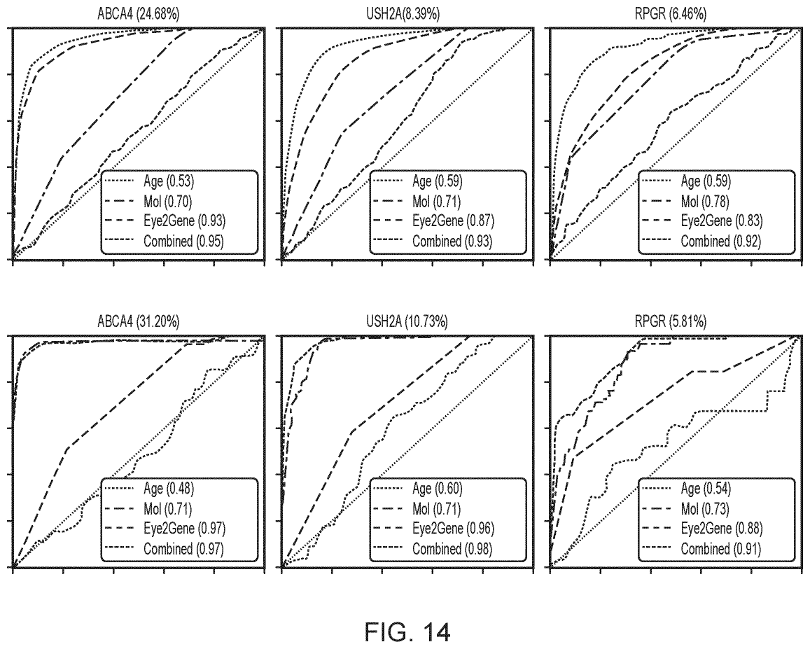

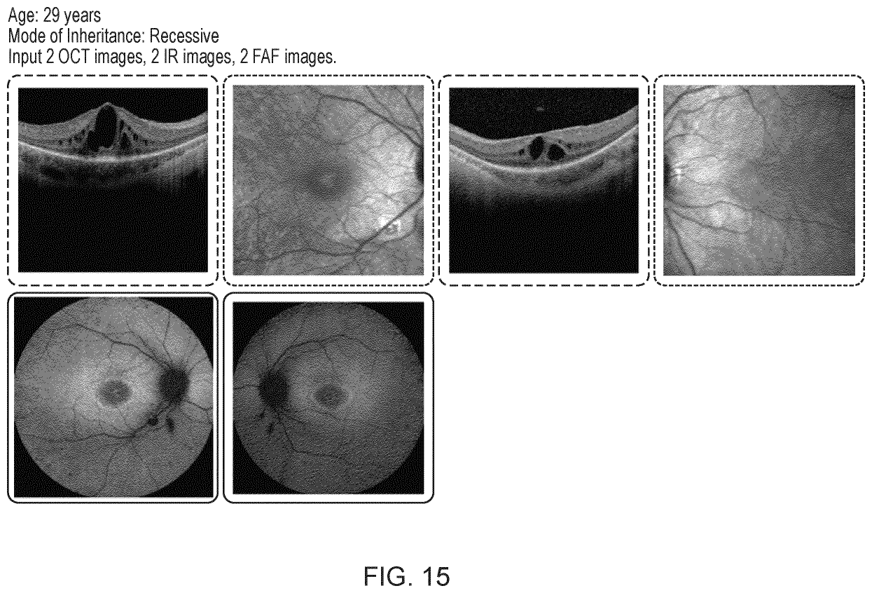

FIG. 14 is a collection of ROC curves for linear classifiers trained on different combinations of features (Age, Mode of Inheritance, and model output of the trained ensemble network (“Eye2Gene”)) using the cross-validation data (top row) and on the internal test dataset (bottom row). For the cross-validation data, a linear model is trained on four folds and tested on the remaining fold; the ROC curves shown are the average over the five folds, with the shaded region being one standard error away from the mean. For the internal test dataset, a linear model is trained on the development set and tested on the internal test dataset. In both cases, one can see the improved conferred by refining the output of the trained ensemble network with further information.

The skilled reader will appreciate that other patient characteristics may be integrated with the classifier to further refine ensemble network outputs. For instance, the patient's ethnic background may be additionally or alternatively encoded for classification. Further, the skilled reader will appreciate that classifiers implementing other algorithms, such as logistic regression with regularisation, may be used.

### Packaging Retinal Scan Image Classification Method

The retinal scan image classification method described herein may be used to assist clinicians in diagnosing IRD patients. The ensemble network may be deployed, for example, as a webapp online. That is, the trained ensemble network may be accessible as an online app.

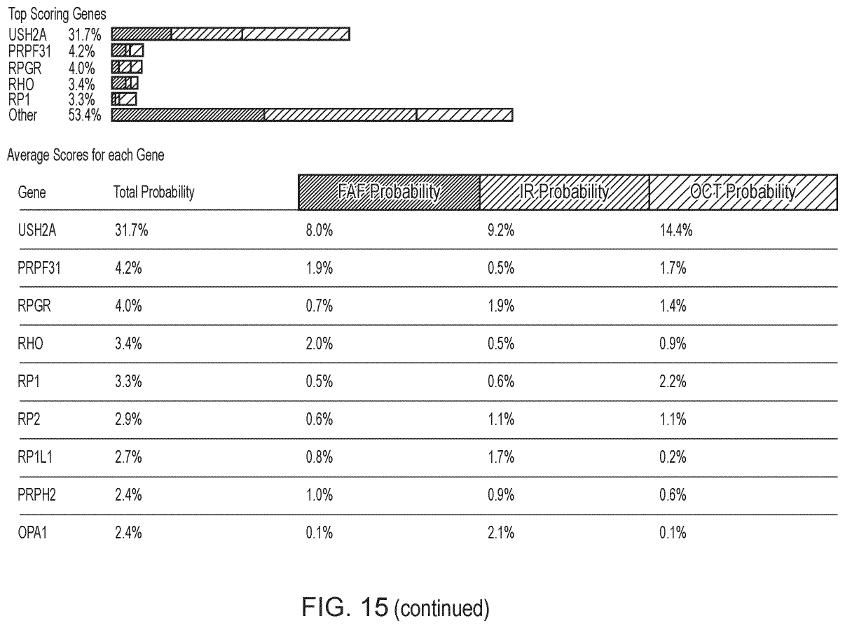

FIG. 15 is an example of a suitable screen of a graphical user interface (GUI), providing example results to a user following execution of the ensemble network. The screen (and any other screen) may be presented to a user on a handheld device or a computer screen. To use the example app, users upload a series of scans (for instance, as PNG images) and may input basic case information such as age at presentation and MOI, if known. In this case, an age of 29 and a MOI of “recessive” is used. The user is then asked to specify the type of scan (choosing, in this example, between SD-OCT, 55-degree fAF, and 30-degree IR) for each image. These images are then passed to the trained ensemble network (to one or all trained CNNs of the relevant imaging modality), which outputs a set of prediction scores for each of the 36 genes on each of the input scans. This information is then aggregated (or ensembled) into an overall prediction score for that case and presented to the user.

In this example, the user is presented with a bar chart of the top 5 genes as predicted by the ensemble network, along with the model probability score for each gene. These predictions are broken down into the contributions of the three different modalities, which are displayed as different colours or shades on the bar-graph. The input images, color-coded by modality (here demonstrated with solid lines bounding FAF scans, long-dashed lines bounding OCT scans, and short-dashed lines bounding IR scans), and patient information are included at the top of the display. A full breakdown, with predicted probabilities for all 36 genes, is presented in the table below the bar graph. A link in the top right of the table takes the user to a breakdown of ensemble network's predictions on each of the uploaded scans.

### Applicability to Other Eye Diseases

The above examples provide methods and networks for retinal scan image classification, which classify retinal scans on the basis of gene abnormalities. Different imaging modalities highlight different aspects of retinal diseases by offering different “views” and therefore have the potential of contributing different pieces of complementary evidence towards an accurate disease diagnosis. Therefore, analysing multiple imaging modalities in parallel offers greater insight into the pathology of disease than examining one at a time.

Hence the multi-modal approach disclosed herein is applicable to all eye diseases (genetic and non-genetic), which includes retinal conditions, age-related macular degeneration (AMD), diabetic retinopathy (DR), glaucoma, retinopathy of prematurity (ROP) and vein occlusions, disease of the optic disc, such as glaucoma and optic neuropathy, as well as conditions affecting the cornea such as keratoconus, corneal dystrophies, and keratitis.

As an example, consider AMD. The blue autofluorescence imaging modality highlights distribution of lipofuscin in the retinal pigment epithelium at a fairly wide view of 50 degrees. Lipofuscin accumulation is a by-product of intracellular aging and therefore an important biomarker for retinal aging as in AMD.

Optical Coherence Tomography (OCT) imaging modality highlights the different layers in a cross-section of the retina and therefore may detect any abnormalities such as accumulation of retinal fluid, as is the case in AMD.

Infrared imaging highlights abnormalities in retinal vasculature so may identify abnormal vascularisation as happens in AMD.

This can be complemented by OCT angiography imaging that may highlight abnormal vascularisation in different layers of the retina.

Depending on where the disease is occurring and its stage, the above imaging modalities may also be combined with wider angle imaging to capture changes in the peripheral retina.

Thus, in the case of AMD retinal scan classification, one may employ methods according to the invention in respect of four (or five) imaging modalities and produce ensembled probabilities of the presence of AMD within the retinal scan.

Naturally, a similar multimodal approach may be used for identifying cases of glaucoma, where the disease may be detectable both in the cornea, the optic disc, the peripheral retina and may be detected in OCT through thinning of the retinal nerve fibre layer.

### Hardware

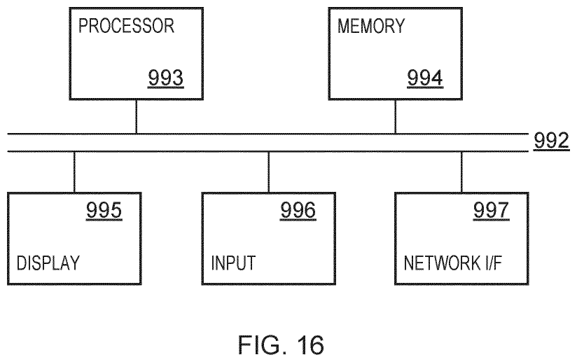

FIG. 16 is a block diagram of a computing device, such as a data storage server, which embodies the present invention and which may be used to implement aspects of the methods for retinal scan image classification, as described herein. The computing device comprises a processor 993, and memory 994. Optionally, the computing device also includes a network interface 997 for communication with other computing devices.

For example, an embodiment may be composed of a network of such computing devices. Optionally, the computing device also includes one or more input mechanisms such as keyboard and mouse 996, and a display unit such as one or more monitors 995. The components are connectable to one another via a bus 992.

The memory 994 may include a computer readable medium, a term which may refer to a single medium or multiple media (e.g., a centralised or distributed database and/or associated caches and servers) configured to carry computer-executable instructions or have data structures stored thereon. Computer-executable instructions may include, for example, instructions and data accessible by and causing a general-purpose computer, special purpose computer, or special purpose processing device (e.g., one or more processors) to perform one or more functions or operations. Thus, the term “computer-readable storage medium” may also include any medium that is capable of storing, encoding, or carrying a set of instructions for execution by the machine and that cause the machine to perform any one or more of the methods of the present disclosure. The term “computer-readable storage medium” may accordingly be taken to include, but not be limited to, solid-state memories, optical media, and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

The processor 993 is configured to control the computing device and to execute processing operations, for example executing code stored in the memory 994 to implement the various different functions of the retinal scan image classification method or the retinal scan image classification training process(es), as described here and in the claims.

The memory 994 may store data being read and written by the processor 993, for example data from training or classification tasks executing on the processor 993. For instance, the memory 994 may store information concerning the chosen architecture(s) of each CNN and may store weights for implementation with the architecture(s).

As referred to herein, a processor 993 may include one or more general-purpose processing devices such as a microprocessor, central processing unit, GPU, or the like. The processor 993 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 993 may also include one or more special-purpose processing devices such as an application specific integrated circuit (ASIC), a field programmable gate array (FPGA), a digital signal processor (DSP), network processor, or the like. In one or more embodiments, a processor 993 is configured to execute instructions for performing the operations and steps discussed herein.

The network interface (network I/F) 997 may be connected to a network, such as the Internet, and is connectable to other computing devices via the network. The network I/F 997 may control data input/output from/to other apparatuses via the network.

Methods embodying aspects of the present invention may be carried out on a computing device such as that illustrated in FIG. 16. Such a computing device need not have every component illustrated in FIG. 16 and may be composed of a subset of those components. A method embodying aspects of the present invention may be carried out by a single computing device in communication with one or more data storage servers via a network or by a plurality of computing devices operating in cooperation with one another. Cloud services implementing computing devices may be deployed.

1. A computer-implemented method of retinal scan image classification, the method comprising:
receiving an input dataset comprising at least one retinal scan, the retinal scan acquired using one of a plurality of imaging modalities;
passing the input dataset through an ensemble network, the ensemble network comprising a plurality of convolutional neural networks, wherein
each convolutional neural network is configured to classify retinal scans acquired using one of the plurality of imaging modalities based on a plurality of eye pathoses, each convolutional neural network producing probabilities of the presence of each of the eye pathoses within retinal scans, and
the retinal scan is processed using a convolutional neural network corresponding to the imaging modality used for acquisition of the retinal scan; and
producing ensembled probabilities of the presence of each of the eye pathoses within the retinal scan.

2. The method of retinal scan image classification of claim 1, wherein
the input dataset comprises a plurality of retinal scans, each retinal scan acquired using one of the plurality of imaging modalities, and
each retinal scan is processed using a convolutional neural network corresponding to the imaging modality used for acquisition of the retinal scan.

3. The method of retinal scan image classification of claim 1, wherein the plurality of imaging modalities comprises any or all of the following: Fundus autofluorescence ((FAF)); infrared ((IR)); and spectral-domain optical coherence tomography ((SD-OCT)).

4. The method of retinal scan image classification of claim 1, wherein the plurality of eye pathoses are genetic abnormalities.

5. The method of retinal scan image classification of claim 4, further comprising passing the ensembled probabilities through a linear classifier, wherein the linear classifier is configured to refine the ensembled probabilities based on subject age and/or mode of inheritance ((MOI) of genetic abnormalities.

6. The method of retinal scan image classification of claim 1, wherein
the ensemble network comprises k convolutional neural networks per imaging modality, and
the retinal scan is processed using k convolutional neural networks, each convolutional neural network corresponding to the imaging modality used for acquisition of the retinal scan, the method further comprising:
averaging the probabilities of the presence of each of the eye pathoses within the retinal scans from each of the k convolutional neural networks.

7. The method of retinal scan image classification of claim 1, further comprising outputting, when the estimation of the presence of each of the eye pathoses within the retinal scan is below a predefined confidence threshold, a prompt to reacquire the retinal scan.

8. The method of retinal scan image classification of claim 1, further comprising processing the output dataset to provide a classification of genetic cause of eye pathosis.

9. (canceled)

10. A computer-implemented method of training an ensemble network for retinal scan image classification, comprising:
receiving an input training dataset comprising training retinal scans labelled with acquisition imaging modality and known eye pathoses, each training retinal scan acquired using one of a plurality of imaging modalities;
splitting the training retinal scans into a plurality of sets of training retinal scans, each set of training retinal scans comprising training retinal scans acquired using the same imaging modality;
for each set of training retinal scans, training a convolutional neural network so as to minimise errors between predicted classifications of a plurality of eye pathoses and the known eye pathoses; and
for each convolutional neural network, outputting the trained convolutional neural network weights.

11. The method of training an ensemble network for retinal scan image classification of claim 10, wherein training the convolutional neural networks uses k-fold cross validation, such that the method comprises, for each set of training retinal scans, training k convolutional neural networks.

12. The method of training an ensemble network for retinal scan image classification of claim 10, further comprising augmenting the input training dataset by any or all of the following: rotating; flipping; zooming; cropping; adjusting brightness; blurring; and adding noise.

13. The method of training an ensemble network for retinal scan image classification of claim 10, further comprising pre-processing the input training dataset by any or all of the following: filtering by median pixel intensity; filtering by noise level; filtering by identification of image artefacts; and filtering by BRISQUE score.

14. A data processing apparatus comprising a memory and a processor configured to perform the method of claim 1.

15. A computer program comprising instructions which, when the program is executed by a computer, cause the computer to carry out the method of claim 1.

16. (canceled)

17. A data processing apparatus comprising a memory and a processor configured to perform the method of claim 10.

18. A computer program comprising instructions which, when the program is executed by a computer, cause the computer to carry out the method of claim 10.
